(19) United States
(12) Patent Application Publication
(10) Pub. No.: US 2004/0157330 A1
(43) Pub. Date: Aug. 12, 2004

(54) Compositions and methods for targeted biological delivery of molecular carriers

(75) Inventors: Philip L. Sheridan, San Diego, CA (US); Steven J. Chapin, San Francisco, CA (US); Derrick L. Domingo, San Diego, CA (US)

Correspondence Address: FOLEY & LARDNER, P.O. BOX 80278, SAN DIEGO, CA, 92138-0278, US

(73) Assignee: Arizeke Pharmaceuticals, Inc.

(21) Appl. No.: 10/754,247

(22) Filed: Jan. 9, 2004

Related U.S. Application Data
(60) Provisional application No. 60/439,372, filed on Jan. 9, 2003.

(51) Int. Cl. C12N 15/85

(52) U.S. Cl. 435/455

(57) ABSTRACT

The present invention relates generally to compositions adapted to provide delivery of therapeutic, diagnostic, prophylactic, or imaging molecules across polarized cells, and methods of their use. By associating two or more targeting elements in or with the composition, one of which binds to a first cell surface component, and another that binds to a second component of the cell that is not initially available to the composition in an amount sufficient to promote effective delivery of all or a portion of the composition into and/or across polarized cells, the compositions of the present invention can provide enhanced bioavailability of medically-relevant moieties.

### RELATED APPLICATIONS

This application claims the benefit of U.S. Provisional Application No. 60/439,372, filed Jan. 9, 2003, the contents of which are incorporated herein in their entirety.

### FIELD OF THE INVENTION

The invention relates to compositions and methods for the delivery of molecules into, through, out of and around, epithelial cells and layers and, optionally, to an intracellular location.

### BACKGROUND OF THE INVENTION

The following description of the background of the invention is provided simply as an aid in understanding the invention and is not admitted to describe or constitute prior art to the invention.

Certain modes for delivering medically important molecules (e.g., oral, nasopharyngeal, oropharyngeal, pulmonary, buccal, sublingual, ocular, mucosal, vaginal, or rectal delivery modes) require that the molecule(s) of interest be delivered across cells (e.g., epithelial cells) that have two distinct surfaces: an apical surface, which is exposed to the aqueous or gaseous medium in which the molecule(s) of interest are delivered to the subject; and an opposing basolateral (a.k.a. basal lateral) side that rests upon and is supported by an underlying basement membrane, and that can provide access to the subject's general circulation. Tight junctions between adjacent epithelial cells separate the apical and basolateral sides of an individual epithelial cell.

Epithelial cells are said to be “polarized;” that is, they are capable of generating gradients between the compartments they separate due to these distinct surfaces having distinct transport and permeability characteristics. (for reviews, see Knust, Curr. Op. Genet. Develop. 10:471-475, 2000; Matter, Curr. Op. Genet. Develop. 10:R39-R42, 2000; Yeaman et al., Physiol. Rev. 79:73-98, 1999). For example, the apical side often contains microvilli for the adsorption of substances from the lumen, and, in ciliated cells, cilia are found on the apical membrane. As another example, the Na⁺/K⁺-ATPase pump is characteristically found only on the basolateral membrane. Because such cells contain pathways that can traffic molecules in a “directional” fashion (e.g., from apical to basolateral), the ability of medically important molecules to “ride” such pathways provides an attractive means to increase bioavailability of such molecules.

Molecules are trafficked into, out from and within a cell by various means. “Active transport” is a general term for the energy-dependent carriage of substances across a cell membrane. “Endocytosis” is a general term for the process of cellular internalization of molecules, i.e., processes in which cells take in molecules from their environment, either passively or actively. “Exocytosis” is a general term for processes in which molecules are passively or actively moved from the interior of a cell into the medium surrounding the cell. “Transcytosis” is a general term for processes in which molecules are transported from one surface of a cell to another. “Paracytosis” is a general term for processes in which molecules are transferred through the interstices between cells, often past tight junctions. “Forward transport” refers to transport in a basolateral to apical direction, while “reverse transport” refers to transport in an apical to basolateral direction.

A number of general methods have been described for delivering medically important molecules, including small molecules, nucleic acids, and/or protein or peptide compositions, in an effort to improve bioavailability and/or to target delivery to particular locations within the body. Such methods include the use of prodrugs, encapsulation into liposomes or other particles, coadministration in uptake enhancing formulations, and targeting to specific tissues. For review see, e.g., Critical Reviews in Therapeutic Drug Carrier Systems, Stephen D. Bruck, ed., CRC Press, 1991. In particular, a number of particulates for the delivery of bioactive substances have been disclosed. Such particles are intended to enhance efficacy, e.g., by avoiding losses in activity caused by enzymatic degradation, protecting from pH extremes, providing a hydrophobic environment for poorly soluble molecules, and/or by enhancing uptake characteristics, etc. See, e.g., U.S. Pat. No. 5,702,727; U.S. Pat. No. 5,620,708; U.S. Pat. No. 5,607,691; U.S. Pat. No. 4,610,896; U.S. Pat. No. 5,149,794; U.S. Pat. No. 6,197,349; U.S. Pat. No. 6,159,502; and U.S. Pat. No. 5,785,976. Such particles may act by (1) decreasing exposure to GI tract luminal proteases (following oral delivery), (2) decreasing exposure to complement defense components in the blood (during intravenous delivery), (3) minimizing dilution effects and/or inactivation of cargo proteins and/or nucleic acids due to binding to non-productive cell types, and (4) minimizing the amount of purified proteins and/or nucleic acids at sites distant from the site of interest, thereby minimizing potentially harmful toxic effects.

Active transport, endocytosis, exocytosis, transcytosis and paracytosis may involve or be mediated by receptors, molecules that are at least partially displayed on the surface of cells. Receptors have varying degrees of specificity; some are specific for a single molecule (e.g., a receptor specific for epidermal growth factor; or a receptor that specifically recognizes Ca⁺⁺); some are semi-specific (e.g., a receptor that mediates the cellular internalization of many members of a family of cellular growth factors, or a receptor that recognizes Ca⁺⁺, Mg⁺⁺ and Zn⁺⁺); or of limited specificity (e.g., a receptor that mediates the cellular internalization of any phosphorylated protein, or a receptor that recognizes any divalent cation). Other types of molecules that can cause or influence the entry of molecules into cells include, e.g., cellular pores, pumps, and coated pits. Pores such as gated channels and ionophores form a channel that extends through the cellular membrane and through which certain molecules can pass. Cellular pumps exchange one type of molecule within a cell for another type of molecule in the cell's environment. Coated pits are depressions in the cellular surface that are “coated” with bristlelike structures and which condense to surround external molecules; the condensed coated pits then “pinch off” to form membrane-bounded, coated vesicles within the cell.

A typical molecule that mediates forward transcytosis is the polymeric immunoglobulin receptor, or “pIgR,” which serves to convey protective antibodies (IgA and IgM immunoglobulins) from the circulatory system to the lumen of an organ. In forward transcytosis, pIgR molecules displayed on the basolateral side of the cell bind IgA molecules from the tissue side (e.g., in the blood or other fluid to which the basolateral surface is exposed), and pIgR:IgA complexes are then endocytosed, i.e., taken up into the cell and into a vesicle. The pIgR:IgA complexes are transported to the apical side of the cell, where they are displayed on the cell surface. Delivery of IgA into the lumen occurs when the pIgR portion of a pIgR:IgA complex is removed from the cell surface, e.g., by proteolysis. This event separates the pIgR molecule into two components: the “secretory component” (SC), which is released into the lumen, and which remains bound to IgA in order to protect IgA from degradation, and the “stalk,” which remains displayed, at least temporarily, on the apical surface of the cell. In certain biological environments, a third region, the “B region,” is removed with the secretory component by initial cleavage, but removed from secretory component by further proteolytic processing to provide “mature secretory component.”

Surprisingly, ligands bound to molecules that mediate forward transcytosis (i.e. in the basolateral to apical direction) displayed on the apical side of a cell can undergo reverse transcytosis; that is, transcytosis in the opposite direction, (i.e., from the apical side of a cell to its basolateral side). In reverse transcytosis, pIgR molecules or portions thereof move from the apical surfaces of cells that line the lumen of an organ to the basolateral surfaces of these cells. pIgR-mediated reverse transcytosis may be used to deliver agents from a lumen (e.g., the interior of the gut or the airways of the lung) to the circulatory system or some other interior system, organ, tissue, portion or fluid of the body including by way of non-limiting example the lymphatic system, the vitreous humor, blood, cerebrospinal fluid, etc. A compound having an element that binds to a portion of pIgR that undergoes reverse transcytosis could, due to its association with the pIgR stalk, be carried to the basolateral side of a cell, where it would be contacted with and/or released into the bloodstream.

The polymeric immunoglobulin receptor (pIgR) is reviewed by Mostov and Kaetzel, Chapter 12 in: Mucosal Immunology, Academic Press, 1999, pages 181-211 (1999). U.S. Pat. No. 6,020,161 to Wu et al. is drawn to pIgR polypeptides and polynucleotides that encode pIgR polypeptides. U.S. Pat. No. 5,484,707 to Goldblum et al. is drawn to methods for monitoring organ rejection in an animal based on the concentration of the free secretory component of (SC) pIgR. Published PCT patent applications WO 98/30592 and WO 99/20310, both to Hein et al., and U.S. Pat. No. 6,045,774 to Hiatt et al., are drawn to synthetic proteins that mimic IgA molecules and are thus associated with the proteolytically generated secretory component (SC) of pIgR. U.S. Pat. No. 6,072,041 to Davis et al. is drawn to fusion proteins that are directed to the secretory component of pIgR. Ferkol et al., Am. J.Respir. Crit. Care Med. 161:944-951, 2000, is stated to describe a fusion protein consisting of a single-chain variable region fragment directed to the secretory component (SC) of human pIgR and an human alpha-(1)-antitrypsin. U.S. Pat. No. 6,042,833 to Mostov et al. is drawn to a method by which a ligand that binds to a portion of a pIgR molecule is thereby internalized into, or transported across, a cell expressing or displaying pIgR. U.S. Pat. No. 6,083,741, to Hart et al., entitled “Internalization of DNA, Using Conjugates of Poly-lysine and an Intergrin Receptor Ligand,” combines this technique with the use of an integrin receptor ligand. Zhang et al. (Cell 102:827-837, 2000) states that pIgR translocates bacteria (specifically, Streptococcus pneumoniae) across nasopharyngeal epithelial cells in the apical to basolateral (reverse) direction.

U.S. patent applications related to the present application include U.S. Provisional Patent Application No. 60/199,423 entitled “Compositions Comprising Carriers and Transportable Complexes,” filed Apr. 23, 2000; PCT/US01/09699, entitled “Ligands Directed to the Non-Secretory Component, Non-Stalk Region of pIgR and Methods of Use Thereof,” filed Mar. 27, 2000; and PCT/US01/30832 entitled “Compositions and Methods for Identifying, Characterizing, Optimizing and Using Ligands to Transcytotic Molecules,” filed Oct. 10, 2001.

Each publication and patent application in the foregoing Background section is hereby incorporated by reference in its entirety, including all tables, figures, and claims.

### SUMMARY OF THE INVENTION

The present invention relates generally to compositions adapted to provide delivery of therapeutic, diagnostic, prophylactic, or imaging molecules (referred to herein as “medically-relevant moieties”) into and/or across polarized cells, and methods of their use. By associating two or more targeting elements in or with the composition, one of which binds to a first cell surface component, and another that binds to a second component of the cell that is not initially available to the composition in an amount sufficient to promote effective delivery of all or a portion of the composition into and/or across polarized cells, the compositions of the present invention can provide enhanced bioavailability of medically-relevant moieties. Such compositions are “bispecific” (or “polyspecific” if additional targeting elements are employed in the same composition) in that they bind more than one molecular target.

In various embodiments, the first cell surface component promotes paracellular transport, active transport, endocytosis and/or transcytosis of all or a portion of the composition into a cell from the cell surface (e.g., the apical surface and/or the basolateral surface), and the second cellular component is an intracellular component that promotes delivery of all or a portion of the composition to another cell surface (e.g., the basolateral surface if the first surface is the apical surface, or vice versa).

In other embodiments, the first cell surface component promotes paracellular transport, active transport, endocytosis and/or transcytosis of all or a portion of the composition into a cell from the cell surface (e.g., the apical surface and/or the basolateral surface), and the second cellular component is an intracellular component that promotes retention of all or a portion of the composition within the cell. In preferred embodiments, the present invention can promote delivery into a cellular organelle (e.g., the nucleus, mitochondria, endoplasmic reticulum, ER-Golgi intermediate compartment, peroxisome, lysosome, aggresome, endosome, etc., of a cell) and/or into the cytoplasm.

In still other embodiments, the first cell surface component promotes delivery of all or a portion of the composition from one cell surface environment (e.g., a mechanical barrier surface such as a mucus layer on polarized epithelium) to a second cell surface environment (e.g., the apical or basolateral cell surface), and the second cellular component promotes paracellular transport, active transport, endocytosis and/or transcytosis of all or a portion of the composition into and/or across a cell from the cell surface.

The skilled artisan will understand that additional targeting elements may be made a part of the composition as desired. For example, a first targeting element may target a first cell surface component that promotes delivery of all or a portion of the composition from one cell surface environment to a second cell surface environment, where a second targeting element targets a second cellular component that promotes paracellular transport, active transport, endocytosis and/or transcytosis of all or a portion of the composition into a cell from the cell surface, where a third targeting element may target a third cellular component that is an intracellular component that promotes delivery of all or a portion of the composition to another cell surface.

Preferably, the medically relevant moieties are one or more molecules independently selected from the group consisting of a protein (which can be one or more polypeptide chains linked covalently or noncovalently) a polypeptide, an enzyme, an antibody, an antibody fragrnent, a single-chain variable region fragment, a glycoprotein, a glycopeptide, a small molecule, an oligonucleotide, an oligosaccharide, a polysaccharide, a cyclic polypeptide, a peptidomimetic, and an aptamer. Such medically-relevant moieties can be naturally occurring or synthetically constructed and/or designed. The compositions of the present invention can be used to deliver such payload molecules via common routes of delivery, such as an oral, nasopharyngeal, oropharyngeal, pulmonary, buccal, sublingual, mucosal, vaginal, transcutaneous, or rectal route.

Thus, in a first aspect, the present invention relates to compositions comprising (i) one or more medically-relevant moieties associated with (ii) a first targeting element that binds to a first cell surface component, and (iii) a second targeting element that binds to a second component of the cell that is not initially available to the composition. In various embodiments, each of the three components of the composition (i-iii) can be associated to the one or more other portions by a variety of independently selected methods known to those of skill in the art, including one or more of the following: covalent attachment of components to one another, electrostatic attachment of components to one another, hydrophobic attachment of components to one another, and incorporation or entrapment of one or more components within or onto a particle or capsule, preferably a microparticle, nanoparticle, microcapsule, or nanocapsule.

In preferred embodiments, the first targeting element specifically binds to a cell surface component that promotes paracellular transport, active transport, endocytosis and/or transcytosis of all or a portion of the composition into a cell from the apical surface, and the second targeting element specifically binds to an intracellular component that promotes basolateral delivery of all or a portion of the composition.

In other preferred embodiments, the first targeting element specifically binds to a cell surface component that promotes paracellular transport, active transport, endocytosis and/or transcytosis of all or a portion of the composition into a cell from the cell surface, and the second targeting element specifically binds to an intracellular component that promotes retention of all or a portion of the composition within the cell.

In still other preferred embodiments, the first targeting element specifically binds to a cell surface component that promotes delivery of all or a portion of the composition from one cell surface environment (e.g., a mechanical barrier surface such as a mucus layer on polarized epithelium) to a second cell surface environment (e.g., the apical or basolateral cell surface), and the second targeting element specifically binds to a cellular component that promotes paracellular transport, active transport, endocytosis and/or transcytosis of all or a portion of the composition into and/or across a cell from the cell surface.

Preferably, the cell surface component to which a targeting element binds is present on epithelial cells, most preferably enterocytes; the cell surface component is present on endothelial cells; the first targeting element and the medically relevant moiety are covalently bound to one another; the first targeting element and the medically relevant moiety are noncovalently bound to one another; the first targeting element and the second targeting element are covalently bound to one another; the first targeting element and the second targeting element are noncovalently bound to one another; the second targeting element and the medically relevant moiety are covalently bound to one another; the second targeting element and the medically relevant moiety are noncovalently bound to one another; and/or the first and second targeting elements are independently selected from the group consisting of an antibody, an antibody fragment, a single-chain variable region fragment, a small molecule, an oligonucleotide, an oligosaccharide, a polysaccharide, a cyclic polypeptide, a peptidomimetic, and an aptamer.

Also preferably, the cell surface component to which a targeting element binds is selected from the group consisting of pIgR, pIgR stalk, an apolipoprotein (e.g., apolipoprotein A1, A2, A3, A4, A5, B, C1, C2, C3, C4, D, and/or E), a cytokine receptor, a Toll- or Toll-like receptor, a receptor tyro sine kinase, a scavenger receptor, a GPI-linked protein, a glycolipid, a glycosphingolipid, a ceramide, a cerebroside, transferrin receptor, transferrin bound to transferrin receptor, apo-transferrin bound to transferrin receptor, vitamin B12 receptor, FcRn, members of the PGDF and VEGF receptor families (e.g., Flt-1, Flk-1, Flt-4), aquaporin, high density lipoprotein binding proteins (e.g., ATP binding casette protein-1, scavenger receptor-BI), a cadherin (e.g., E-cadherin, N-cadherin, P-cadherin, R-cadherin, K-cadherin, and/or OB-cadherin), and low density lipoprotein receptor; the intracellular component to which a targeting element binds is selected from the group consisting of pIgR, pIgR stalk, an apolipoprotein (e.g., apolipoprotein A1, A2, A3, A4, A5, B, C1, C2, C3, C4, D, and/or E), transferrin receptor, transferrin bound to transferrin receptor, apo-transferrin bound to transferrin receptor, vitamin B12 receptor, FcRn, members of the PGDF and VEGF receptor families (e.g., Flt-1, Flk-1, Flt-4), aquaporin, high density lipoprotein binding proteins (e.g., ATP binding casette protein-1, scavenger receptor-BI), a cadherin (e.g., E-cadherin, N-cadherin, P-cadherin, R-cadherin, K-cadherin, and/or OB-cadherin), and low density lipoprotein receptor; and/or the medically-relevant moiety is selected from the group consisting of a polypeptide, an antibody, an antibody fragment, a single-chain variable region fragment, a small molecule, an oligonucleotide, an oligosaccharide, a polysaccharide, a cyclic polypeptide, a peptidomimetic, and an aptamer.

In particularly preferred embodiments, the specific binding between the first targeting element and the cell surface component has a dissociation constant (K_{d}), measured at a pH provided by the environment to which the apical surface of a target cell is exposed, that is less than the K_{d} measured at a pH provided by an endosomal compartment to which the complexed first targeting element/cell surface component is exposed upon transport into the cell; and/or the specific binding between the second targeting element and the intracellular component has a dissociation constant (K_{d}), measured at a pH provided by an endosomal compartment within the cell, that is less than the K_{d} at a pH provided by the environment to which the basolateral surface of a target cell is exposed. Under such conditions, the composition of the present invention can bind to the cell surface component, resulting in uptake of all or a portion of the composition, but the composition can be released from the cell surface component upon internalization; similarly, the composition can bind to the second targeting element within the endosome, but the composition can be released upon transport to the basolateral surface.

In other particularly preferred embodiments, the binding affinity of the first targeting element for its binding partner (e.g., the cell surface component) is lower than the binding affinity between the second targeting element and its binding partner (e.g., the intracellular component).

In those embodiments in which covalent association of a component to another component (or to a particle or capsule) is desired, appropriate associative interactions can be formed in a variety of ways known to those of skill in the art. In preferred embodiments, amino acid residues present in the natural sequence of a first protein can be directly covalently linked to amino acid residues in the natural amino acid sequence of a second protein as in, e.g., a disulfide bridge; in such methods, mutant amino acids useful for forming covalent linkages (e.g., cysteine residues) can be introduced into one or more proteins using molecular genetics, or, in the case of synthetic oligopeptides, directly during the in vitro synthesis thereof. In other preferred embodiments, natural or mutant amino acid sequences present in isolated proteins can be “derivatized” (i.e, chemically modified in vitro) so as to include chemical groups not present in natural amino acids but useful for the chemical conjugation of oligopeptides, polypeptides, and proteins in a related methodology, unnatural amino acids having moieties useful for chemical conjugation are introduced into oligopeptides or peptidomimetics during their synthesis in vitro. In still other preferred embodiments, a cross-linking reagent (a.k.a. “a cross-linker”) can be used to covalently link components to each other. Such bifunctional linkers can be homobifunctional (wherein both “arms” of the linker are the same chemical moiety) or heterobifunctional (wherein each of the two “arms” is a different chemical moiety than the other).

When the noncovalent association of a component to another component (or to a particle or capsule) is desired, appropriate associative interactions that may be employed include, but are not limited to, antibody-antigen, receptor-hormone, avidin-biotin pairs, streptavidin-biotin, metal-chelate, small molecule/polynucleotide (see, e.g., Dervan, Bioorg. Med. Chem. 9: 2215-35 (2001); Zahn and Dervan, Bioorg. Med. Chem. 8: 2467-74 (2000)); polynucleotide/complementary polynucleotide (e.g. dimeric and trimeric helices), aptamer/small molecule, aptamer/polypeptide, coiled-coil, and polynucleotide/polypeptide (e.g. zinc finger, helix-turn-helix, leucine zipper, and helix-loop-helix motifs that bind to DNA sequences).

By way of non-limiting example, multivalent complexes or compounds of the invention comprise two, three, four, or more different targeting elements that specifically bind to different cell surface components and/or to different intracellular components.

The term “therapeutic composition” as used herein refers to a composition for treating a disease, or one or more symptoms thereof, in a human or non-human animal subject.

The term “diagnostic composition” as used herein refers to a composition for identifying the presence or absence of one or more markers related to the presence or absence of a disease. Preferably, a diagnostic composition comprises an antibody.

The term “prophylactic composition” as used herein refers to a composition for preventing a disease, or one or more symptoms thereof, in a human or non-human animal subject. Most preferably, a prophylactic composition is an immunogenic composition that acts as a vaccine to prevent a disease or symptom of a disease.

The term “imaging composition” refers to a composition for enhancing contrast in an X-ray, MR, CT, nuclear, and/or acoustic (e.g., ultrasound) procedure. Typical particulate imaging compositions are disclosed in, e.g., U.S. Pat. No. 6,251,366; U.S. Pat. No. 6,203,777; U.S. Pat. No. 5,976,500; U.S. Pat. No. 5,928,626; and U.S. Pat. No. 5,670,135, each of which is hereby incorporated by reference in its entirety, including all tables, figures, and claims

The term “therapeutic moiety” as used herein refers to a molecule or portion of a molecule that, when introduced into a living organism, modifies one or more functions of the organism. Preferred therapeutic moieties are small molecules, prodrugs, polypeptides, antibodies, antibody fragments, single-chain variable region fragments, polynucleotides, oligonucleotides, oligonucleotide analogs, oligosaccharides, polysaccharides, cyclic polypeptides, peptidomimetics, and aptamers.

As used herein, the term “small molecule” refers to compounds having molecular mass of less than 3000 Daltons, preferably less than 2000 or 1500, still more preferably less than 1000, and most preferably less than 600 Daltons. Preferably but not necessarily, a small molecule is not an oligopeptide.

As used herein, the term “polypeptide” refers to a covalent assembly comprising at least two monomeric amino acid units linked to adjacent amino acid units by amide bonds. An “oligopeptide” is a polypeptide comprising a short amino acid sequence (i.e., 2 to 10 amino acids). An oligopeptide is generally prepared by chemical synthesis or by fragmenting a larger polypeptide. Examples of polypeptide drugs include, but are not limited to, therapeutic antibodies, insulin, parathyroid hormone, polypeptide vaccines, and antibiotics such as vancomycin. Novel polypeptide drugs may be identified by, e.g., phage display methods.

As used herein, the term “antibody” refers to an immunoglobulin molecule obtained by in vitro or in vivo generation of an immunogenic response, and includes both polyclonal, monospecific and monoclonal antibodies, and antigen binding fragments thereof (e.g., Fab fragments). An “immuriogenic response” is one that results in the production of antibodies directed to one or more proteins after the appropriate cells have been contacted with such proteins, or polypeptide derivatives thereof, in a manner such that one or more portions of the protein function as epitopes.

As used herein, the term “single-chain variable region fragment” or “scFv” refers to a variable, antigen-binding determinative region of a single antibody light chain and antibody heavy chain linked together by a covalent linkage having a length sufficient to allow the light and heavy chain portions to form an antigen binding site. Such a linker may be as short as a covalent bond; preferred linkers are from 2 to 50 amino acids, and more preferably from 5 to 25 amino acids.

As used herein, the term “polynucleotide” refers to molecule comprising a covalent assembly of nucleotides linked typically by phosphodiester bonds through the 3′ and 5′ hydroxyls of adjacent ribose units. An “oligonucleotide” is a polynucleotide comprising a short base sequence (i.e., 2 to 10 nucleotides). Polynucleotides include both RNA and DNA, may assume three-dimensional shapes such as hammerheads, dumbbells, etc., and may be single or double stranded. Polynucleotide drugs can include ribozymes, ribozymes, and polynucleotide vaccines.

As used herein, the term “oligonucleotide analog” refers to a molecule that mimics the structure and function of an oligonucleotide, but which is not a covalent assembly of nucleotides linked by phosphodiester bonds. Peptide nucleic acids, comprising purine and pyrimidine bases linked via a backbone linkage of N-(2-aminoethyl)-glycine units, is an example of an oligonucleotide analog.

The term “polysaccharide” as used herein refers to a carbohydrate comprising 2 or more covalently-linked saccharide units. An “oligosaccharide” is a polysaccharide comprising a short saccharide sequence (i.e., 2 to 10 saccharide units).

As used herein, the term “cyclic polypeptide” refers to a molecule comprising a covalent assembly of monomeric amino acid units, each of which is linked to at least two adjacent amino acid units by amide bonds to form a macrocycle.

As used herein, the term “peptidomimetic” refers to a molecule that mimics the structure and function of a polypeptide, but which is not a covalent assembly of amino acids linked by amide bonds. A peptoid, which is a polymer of N-substituted glycine units, is an example of a peptidomimetic.

The term “aptamer” as used herein refers to polynucleotides that bind to non-polynucleotide target molecules (e.g., a polypeptide or small molecule).

The term “scavenger receptor” as used herein refers to a class of proteins that mediates the uptake of modified forms of lipoproteins, including low density lipoproteins (“LDL”). Cell types such as macrophages, endothelial cells, intestinal epithelial cells, and smooth muscle cells have been shown to have scavenger receptors for modified lipoproteins, and the scavenger receptor family has grown to include cell surface receptors which mediate cholesterol transport by ‘scavenging’ cholesterol from HDL. Scavenger receptors also bind a range of polyanionic ligands other than modified lipoproteins. See, e.g., Platt and Gordon, Chem. Biol. 5: R193-203 (1998); Werder et al., Biochemistry 40: 11643-50 (2001); Zingg et al., Arterioscler. Thromb. Vasc. Biol. 22: 412-17 (2002). Exemplary scavenger receptors include SR-A1/II, which binds aceylated and oxidized LDL, LPS, and bacteria; MARCO, which binds bacteria; CD36, which binds oxidized LDL; CLA-1, which is a human homologue of SR-B1 and which mediates reverese cholesterol transport; CD68, which binds oxidized LDL, LOX-1, which binds oxidized LDL; Galectin, which binds aceylated LDL, oxidized LDL, and advanced glycation endpoint LDL; and SR-PSOX, which binds oxidized LDL.

The term “GPI-linked protein” as used herein refers to a class of eukaryotic proteins that have a glycosylphosphoinositol lipid (GPI) modification at the carboxy-terminal end. The GPI moiety, added posttranslationally to proteins in the endoplasmic reticulum in vivo, that serves as a means of membrane anchoring of a protein to the external plasma membrane. In polarized cells, such as MDCK cells, GPI-linked proteins are preferentially segregated to the apical cell surface, where they may be associated with microdomains known as “rafts.” Rafts, and their GPI-linked contents, can be internalized under certain conditions, such as by antibody-induced crosslinking of GPI-linked proteins. At least a portion of these internalized rafts may be transcytosed by the polarized cells. See, e.g., Verkade et al., J. Cell Biol. 148: 727-39 (1999); Muniz and Riezman, EMBO J. 19: 10-15 (2000).

The term “targeting element” as used herein refers to any molecular structure that is directed to (specifically binds) a molecule to which it is targeted. The term “specifically binds” is not intended to indicate that the targeting element binds exclusively to its intended target. Rather, a targeting element specifically binds if its affinity for its intended target is about 2-fold greater when compared to its affinity for a non-target molecule. Preferably the affinity of the targeting element will be at least about five fold, preferably 10 fold, more preferably 25-fold, even more preferably 50-fold, and most preferably 100-fold or more, greater for a target molecule than its affinity for a non-target molecule. A composition comprising such a targeting element would be referred to as being “adapted to specifically bind” to the target molecule. Preferred targeting elements can be selected from the group consisting of a polypeptide, a recombinant polypeptide, an antibody, an antibody fragment, a single-chain variable region fragment, a small molecule, an oligonucleotide, an oligosaccharide, a polysaccharide, a cyclic polypeptide, a peptidomimetic, and an aptamer, as these terms are defined herein.

Most preferably, a first targeting element as described above is directed to a protein selected from the group consisting of a cell surface receptor, most preferably a pIgR molecule; and a cell surface molecule other than a receptor, most preferably a pIgR stalk molecule, or a pIgR secretory component molecule. Such elements confer the property of being able to undergo apical or basolateral endocytosis, apical or basolateral exocytosis, and/or forward or reverse transcytosis in cells displaying a pIgR molecule, a pIgR stalk molecule, and/or a pIgR secretory component molecule. By way of non-limiting example, the pIgR may be a simian pIgR, a murine pIgR, a rodent pIgR, a rabbit pIgR, a bovine pIgR, or a human pIgR.

The term “cell surface component” as used herein refers to a molecule, or a portion of a molecule, present on an external surface of a cell and that is accessible to targeting moieties placed in contact with the cell. Preferred cell surface components include, but are not limited to, receptors such as pIgR, a scavenger receptor, a GPI-linked protein, transferrin receptor, vitamin B12 receptor, FcRn, integrins low density lipoprotein receptor; cargo carrier fragments such as pIgR stalk, members of the PGDF, FGF, and VEGF receptor families (e.g., Flt-1, Flk-1, Flt-4, FGFR1, FGFR2, FGFR3, FGFR4), and surface antigens.

A cell surface component is said to “promote” transport, active transport, endocytosis, or transcytosis if a composition comprising a targeting element that specifically binds to the cell surface component is transported into, around, or through a cell (depending on the type of transport involved) at a higher rate or to a higher absolute amount compared to a similar composition lacking the targeting element. Similarly, a cellular component is said to “promote delivery” from one region of a cell to a second region if a composition comprising a targeting element that specifically binds to the cellular component is transported from the first region to the second region at a higher rate or to a higher absolute amount compared to a similar composition lacking the targeting element. Preferably, a 2-fold, 5-fold, 10-fold, 100-fold, or 1000-fold increase in rate or amount is obtained.

The term “intracellular component” as used herein refers to a molecule, or a portion of a molecule, present within a cell, either in the cytoplasm or in an organelle. Preferred intracellular components are accessible to targeting moieties present within an endosomal compartment of a cell. Preferred intracellular components include, but are not limited to, receptors such as pIgR, transferrin receptor, vitamin B12 receptor, FcRn, integrins, low density lipoprotein receptor; cargo carrier fragments such as pIgR stalk, members of the PGDF, FGF, and VEGF receptor families (e.g., Flt-1, Flk-1, Flt-4, FGFR1, FGFR2, FGFR3, FGFR4), and surface antigens.

An intracellular component is said to “promote basolateral delivery” if a composition comprising a targeting element that specifically binds to the intracellular component is transported to the basolateral surface of a polarized cell at a higher rate compared to a similar composition lacking the targeting element.

The term “endosome” as used herein refers to a membranous organelle to which molecules internalized by a cell via endocytosis are transferred. The endosomal apparatus within polarized cells can be subdivided into two types of compartments: a “housekeeping endosome” that recycles certain internalized cell surface components (e.g., transferrin receptor and low-density lipoprotein receptor) primarily to the basolateral surface; and a “specialized endosome” from which vesicles targeted to the apical surface leave. These are not typically considered to be discrete compartments; that is, components may flow in both directions between these compartments. Proton pumps within the endosome permit the endosome interior to reach a pH of between 5 and 6.

A targeting element is said to be “cell-specific” if it is directed to a cell surface component or an intracellular component that is exclusively or preferentially displayed on or in a cell of a particular cell type or tissue.

In preferred embodiments, the various components of a composition as described herein are associated with one another such that a portion of the first targeting element that specifically binds to a cell surface component, and a portion of the second targeting element that specifically binds to an intracellular component, is properly exposed to participate in the appropriate binding interaction. This proper exposure may be provided, for example, by binding to another component of the composition (or to a particle or capsule) through a portion of the targeting element that is sufficiently separated from the portion that specifically binds to the cell surface component such that steric hindrance of the binding interaction is avoided. Similarly, a linker can be used between the targeting element and the other components of the composition. Preferably, a targeting element is attached to a linker of between about 5 Å and about 1000 Å, more preferably between about 10 Å and about 500 Å, even more preferably between about 50 Å and about 300 Å, and most preferably between about 75 Å and about 200 Å. The term “about” in this context refers to +/−10% of a given measurement.

The term “particle” as used herein refers to an integral structural element having dimensions of between about 1000 μm and about 1 nm in overall dimension capable of retaining one or more molecules for delivery to a subject (“a payload”). Such particles are preferably porous and/or biodegradable, and most preferably selectively porous and/or biodegradable (i.e., only in certain environments). Preferred particles have physical dimensions compatible with cellular uptake, e.g., about 10 μm to about 10 nm, most preferably about 1 μm to about 5 nm. The term “microparticle” refers to particles from 1 to 1000 μm, while “nanoparticle” refers to particles less than 1 μm in size. The term “about” in this context refers to +/−10% of a given dimension. Such particles include polymeric microparticles, virions, liposomes, lipoprotein particles, lipid emulsions, and lipid suspensions, and may comprise an internal polymer matrix, an internal fluid, or an amorphous or crystalline internal phase. See, e.g., U.S. Pat. No. 6,197,349. Particles may be formulated for topical, ingestible, injectable, and inhaled applications.

The term “capsule” as used herein refers to a subset of particles that are vesicular structural elements having dimensions of between about 1000 μm and about 1 nm in overall dimension capable of retaining one or more molecules for delivery to a subject (“a payload”). One or more molecules for delivery are confined to a central cavity surrounded by an outer shell, such as a polymer or lipid membrane. Preferred capsules have physical dimensions compatible with cellular uptake, e.g., about 10 μm to about 10 nm, most preferably about 1 μm to about 5 nm. The term “microcapsule” refers to capsules from 1 to 1000 μm, while “nanocapsule” refers to capsules less than 1 μm in size. The term “about” in this context refers to +/−10% of a given dimension. Capsules can include “particle-in-particle” and “particle-in-coating” structures. See, e.g., Soppimath et al., J. Controlled Release 70: 1-20 (2001); McPhail et al., Intl. J. Pharmaceutics 200: 73-86 (2000).

The term “anchor moiety” as used herein refers to any molecular structure that may be physically entrapped in a particle or capsule and that is or may be noncovalently bound to a targeting element. For example, an anchor moiety may comprise a first region that is a polypeptide, a nucleic acid, a poly(ethylene oxide), a peptidomimetic, a cyclic peptide, a oligosaccharide, a polysaccharide, an aptamer, or a dextran, that is entrapped within a particle, and a second region that projects from the particle and that binds to a complementary region on a targeting element. Any binding interaction may be employed by the skilled artisan for binding between the anchor moiety and the targeting element. In preferred embodiments, the second region comprises a nucleic acid sequence that is complementary to a sequence in the targeting element; the second region comprises an aptamer that binds to a region present on the targeting element; the second region comprises a region that binds to an aptamer present on the targeting element; the second region comprises an amino acid sequence that forms a coiled-coil domain with an amino acid sequence in the targeting element; and the second region comprises one or more cysteine residues that form one or more disulfide bonds with cysteine residues in the targeting element. These examples are not meant to be limiting.

In another aspect, the present invention relates to compositions comprising (i) one or more medically-relevant moieties associated with (ii) a first targeting element that specifically binds to a cell surface component that promotes paracellular transport, active transport, endocytosis and/or transcytosis of all or a portion of the composition into a cell from the apical surface, and (iii) a second targeting element that specifically binds to a component present on the basolateral surface, or in a fluid to which the basolateral surface is exposed, thereby providing delivery of all or a portion of the composition to the basolateral surface or the fluid. Such compositions are again “bispecific” (or “polyspecific” if additional targeting elements are employed in the same composition) in that they bind more than one molecular target.

In yet another aspect, the present invention relates to methods for providing the therapeutic, diagnostic, prophylactic, or imaging compositions described herein to a subject in need thereof. The compositions of the present invention may be used to deliver medically-relevant moieties to any cell capable of paracellular transport, active transport, endocytosis, and/or transcytosis. Examples of cells that may be targeted by such compositions include epithelial cells (e.g., squamous, transitional cuboidal, stratified, and columnar epithelial cells), most preferably epithelial cells lining the gastrointestinal tract (e.g., enterocytes), the alveolae, the trachea, the nasopharynx, the bronchial tree, the oropharynx, the lung, the vaginal tract, the skin or mucosal surfaces, and/or the rectum; endothelial cells; and endothelial cells, most preferably those cells lining the circulatory and/or the lymphatic systems.

The term “subject” as used herein refers to a human or a non-human animal. Thus, the methods and compositions described herein can be used for both medical and veterinary purposes.

The biological environment presented to such compositions when delivered to a subject can be hostile to medically-relevant moieties present within the composition. In the case of polypeptides and oligonucleotides for example, proteases and nucleases are often present in biological systems (e.g., blood, the gastrointestinal tract, etc.). Additionally, the gastrointestinal tract can expose unprotected medically-relevant moieties to extremes of pH (about pH 1-pH 4.5) It is therefore preferred that the compositions of the present invention protectively retain the medically-relevant moieties prior to cellular uptake, e.g., in a particle or capsule.

The term “protectively retain” is not meant to indicate that all of the medically-relevant moieties originally present in a particle or capsule be bioavailable; instead, the term indicates that at least a percentage of the medically-relevant moieties be bioavailable in a functional form and in an amount sufficient to have its intended biological effect. In preferred embodiments, the compositions of the present invention provide at least about 1% bioavailability, more preferably about 5% bioavailability, still more preferably at least about 10% bioavailability, even more preferably at least about 25% bioavailability, still more preferably at least about 50% bioavailability, and most preferably at least about 75% bioavailability. The term “about” in this context refers to +/−10% of a given percentage (e.g., about 10% means from 9% to 11%).

In preferred embodiments, the particle or capsule is made of a biodegradable material, such that the cargo of the nanoparticle may be released into a cell or into the blood or tissues, thus rendering the biologically-relevant moieties originally present in a particle or capsule bioavailable.

The term “bioavailability” refers to the extent to which a medically-relevant moiety (or the active metabolite of a pro-form of such a moiety, e.g., a prodrug) is available to its site of action. A bioavailability of 5% means that 5% of the moiety delivered to the subject (e.g., by oral delivery, by pulmonary delivery, etc.) is available within the tissues of the subject. In preferred embodiments, a medically-relevant available within the blood, within an interstitial space, within a specific tissue (e.g., lung, kidney, liver, brain, pancreas, tumor tissue, etc.), and/or within tissues protected by the blood-brain barrier. Preferably, bioavailability is measured by determining the percentage of delivered dose appearing in the blood of the subject.

The summary of the invention described above is not limiting and other features and advantages of the invention will be apparent from the following detailed description of the preferred embodiments, as well as from the claims.

### DETAILED DESCRIPTION OF THE INVENTION

The present invention describes compositions for targeted biological delivery of medically relevant molecules, and methods for their production and use. In particular, compositions are formed by associating two or more targeting elements, one of which binds to a first cell surface component, and another that binds to a second component of the cell that is not initially available to the composition in an amount sufficient to promote effective delivery of all or a portion of the composition into and/or across polarized cells. By further associating one or more medically-relevant moieties with the composition, the compositions of the present invention can provide enhanced bioavailability of medically-relevant moieties.

Receptor Switching

Eukaryotic cells have developed a variety of mechanisms for the uptake of extracellular substances and their delivery to membrane surrounded structures, the endosomal compartments. These processes are commonly referred to as endocytosis. In fluid phase endocytosis, small droplets of extracellular fluid (and any material dissolved in it) are surrounded by small portions of the plasma membrane, which invaginate and then bud off to form intracellular transport vesicles. In receptor-mediated endocytosis, specific receptors on the plasma membrane bind tightly to extracellular molecules (ligands). The receptor-ligand complexes are then incorporated into the intracellular transport vesicles.

Transferrin, an iron-binding protein, is a commonly studied example of endocytotic uptake and sorting. Intracellular vesicles containing endocytosed transferrin receptor-transferrin-iron complexes are routed to an early endosome and fuse with this compartment. Within the early endosome sorting occurs and substances are further transported to different subcellular organelles. Molecules destined to be broken down are transported from the early endosome via a late endosome to a lysosome where they are degraded. In the case of transferrin, the relatively low pH (<˜6.0) causes dissociation of iron. Certain receptors are recycled from the early endosome via a recycling endosome back to the plasma membrane for reuse.

In polarized cells, an additional level of complexity is layered upon the already complex endocytosis machinery. The distinct structure and function of polarized cells is provided by the distinct compositions of apical and basolateral membranes. This membrane polarity must be maintained despite continual endocytotic turnover of membrane components. Initially, it was believed that the endosomal network of the apical and basolateral surfaces must be maintained in isolation from one another. It is now understood that this is incorrect, and that the two endosomal networks are interconnected. Nevertheless, the bulk of the material endocytosed from the apical membrane that is recycled back to the cell surface is sorted properly and returns to the apical surface; so too is the material endocytosed from the basolateral membrane.

Certain cells also provide mechanical barriers, such as a mucus layer, that cover and protect exposed surfaces. The natural flow of this mucus layer can prevent pathogens, as well as therapeutic compositions, from effectively gaining access to the endocytotic machinery in the underlying cells. Receptor-mediated endocytosis also occurs at the blood-brain barrier for substances, such as transferrin, insulin, leptin, IGF-I and IGF-II.

Thus, while receptor-mediated endocytosis provides an attractive means for delivery of medically-relevant moieties (e.g., therapeutics) into cells of a subject, its use as a means of delivering medically-relevant moieties across cell layers, and, in preferred embodiments, into the general circulation, may be limited by the very effective means that such cells use to maintain polarity, or by the local environment at a cell surface. As described in detail herein, however, one may take advantage of the interconnections between the various cellular environments (e.g., two interconnected endosomal networks) to direct material taken up at the apical surface for delivery into cells, to the basolateral surface, and/or beyond into the general circulation of a subject. This process is referred to herein as “receptor switching.”

As described in detail herein, the compositions of the present invention comprise one or more targeting moieties, one of which binds to a first cell surface component, and another that binds to a second component of the cell that is not initially available to the composition in an amount sufficient to promote effective delivery of all or a portion of the composition into and/or across polarized cells. The targeting moiety may bind directly to the cell surface component, or in the alternative may bind to a separate ligand that is itself bound to the cell surface component. Thus, in addition to the cell surface receptors described herein, a targeting moiety may also be directed to, and specifically bind, a ligand for any of these receptors.

In exemplary embodiments described hereinafter, a first targeting moiety of the composition specifically binds to a cell surface component that promotes paracellular transport, active transport, endocytosis and/or transcytosis. Once within the cell, the compositions can specifically bind to a second cellular component that preferentially drives delivery of the composition to the basolateral surface of the cell. Preferably, upon binding to a cell surface component, the composition is delivered to the endosomal network, where this second cellular component is present. Upon binding to the intracellular component that promotes delivery to the basolateral surface, the composition is delivered via the recycling function of the endosomal network.

While not a required feature of the present invention, by carefully selecting targeting moieties, and their relative affinities at the pH outside the cell compared to the endosomal compartment, the composition can be “handed off” from the cell surface component at the apical surface to the intracellular component present in the endosome for final delivery to the basolateral surface.

Numerous ligands are known to enter or exit biological systems by binding to a component that mediates transport of the ligand to or from the cell surface. Examples of such ligands include diphtheria toxin, pseudomonas toxin, cholera toxin, ricin, concanavalin A, certain viruses (Rous sarcoma virus, adenovirus, etc.), transferrin, low density lipoprotein, transcobalamin (vitamin B12), insulin, epidermal growth factor, growth hormone, thyroid stimulating factor, calcitonin, glucagon, prolactin, lutenizing hormone, thyroid hormone, platelet derived growth factor, VEGFs, IgA, and IgM. Particularly referred cell surface components include, but are not limited to, receptors such as pIgR, a scavenger receptor, a GPI-linked protein, transferrin receptor, vitamin B12 receptor, FcRn, intergrins, low density lipoprotein receptor; cargo carrier fragments such as pIgR stalk, members of the PGDF, FGF, and VEGF receptor families (e.g., Flt-1, Flk-1, Flt-4, FGFR1, FGFR2, FGFR3, FGFR4), and surface antigens.

This list is not meant to be limiting. Other preferred receptors include scavenger receptors (e.g., CLA-I/SR-B1, CD-36, intrinsic factor, cubilin, megalin, GP 330), p75NTR (Neurotrophin receptor), Leptin receptor, TGF-beta receptor, TGF beta receptor II, reduced folate carrier, Mannose-6-phosphate receptor, CaR (calcium receptor), A2b adenosine receptor, IGF-I receptor, IGF-II receptor, ebnerin (taste), 67 kD laminin receptor, laminin receptor precursor (LRP), TGF-beta receptor III, transcobalamin receptor, HGF-SF (hepatocyte growth factor/scatter factor, c-met) receptor (also known as Met or cMet), CD4 receptor, TGF-beta I receptor, c-erbB (EGF receptor), ASGP-R (asialoglycoprotein receptor), LRP (low density lipoprotein receptor related protein) receptor, CFTR (cyctic fibrosis transmembrane conductance regulator), sucrose isomaltase, receptors for toxins, viruses, and bacteria (e.g., GM1 ganglioside (cholera toxin), Galactosyl ceramide (HIV), receptor for anthrax protective antigen, CD 46 (measles), 85 kD CSL receptor (cryptosporidium), GDlb (E. coli type II temperature sensitive enterotoxin (LTIIa)), GC-C Guanylyl cyclase (E. coli heat stable enterotoxin (STa)), putative Hepatitis A receptor, Toll-like receptor 5 (TLR5)), transporters/exchangers (e.g., PepT 1, ENaC (sodium), GLUT-5, SGLT-1, CaTI (calcium), EcaC (calcium), NHE 3 (Na⁺/H+exchanger)), apolipoproteins (e.g., apolipoprotein A1, A2, A3, A4, A5, B, C1, C2, C3, C4, D, and/or E), aquaporin, high density lipoprotein binding proteins (e.g., ATP binding casette protein-1, scavenger receptor-BI), viral receptors (e.g., coxsakie adenovirus receptor, αv integrins, sialic acid-containing glycoproteins, CD4), and proteases (e.g., epitheliasin, Aminopeptidase N, Dipeptidylpeptidase).

Targeting Molecules for Uptake or Delivery at the Apical Cell Surface

The compositions of the present invention can utilize the same transport mechanisms used by natural ligands to improve bioavailability of molecules. See, e.g., PCT/US01/30832 entitled “Compositions and Methods for Identifying, Characterizing, Optimizing and Using Ligands to Transcytotic Molecules;” filed Oct. 10, 2001. A discussion of immunglobulin transport mediated by pIgR follows as an example of mediated transport useful in the present invention. While the following describes transport across polarized epithelium, the skilled artisan will understand that the present invention can also provide transport across cell layers generally, such as across non-polarized cells or across the blood-brain barrier.

A pIgR molecule has several structurally and functionally distinct regions that are defined as follows. A pIgR molecule binds polymeric immunoglobulins (IgA or IgM) on the basolateral side, and then transports the immunoglobulin to the apical side. Proteolytic cleavage of pIgR takes place on the apical side of an epithelial cell between the SC and the stalk, the former of which remains bound to and protects the immunoglobulins, and the latter of which remains bound to the apical membrane (see “Mucosal Immunoglobulins” by Mestecky et al. in: Mucosoal Immunology, edited by P. L. Ogra, M. E. Lamm, J. Bienenstock, and J. R. McGhee, Academic Press, 1999). Ligands bound to “stalks” displayed on the apical side of a cell can undergo reverse transcytosis, i.e., transcytosis in the opposite direction of forward transcytosis, i.e., from the apical side of a cell to its basolateral side. In reverse transcytosis, pIgR molecules or portions thereof move from the apical surfaces of cells that line the lumen of an organ to the basolateral surfaces of these cells. See, e.g., U.S. Pat. No. 6,072,041, which is hereby incorporated by reference in its entirety, including all tables, figures, and claims.

Extracellular domains 1 through 6 of pIgR molecules from several species are indicated in FIG. 3 of Piskurich et al. (J. Immunol. 154:1735-1747, 1995). In rabbit pIgR, domains 2 and 3 are encoded by a single exon that is sometimes deleted by alternative splicing. A transmembrane domain is also present in pIgR, as is an intracellular domain. The intracellular domain contains signals for transcytosis and endocytosis. Domains of a pIgR molecule that are of particular interest in the present disclosure include but are not limited to domain 5, domain 6, the B region, the stalk, the transmembrane domain, the secretory component, and the intracellular domain.

As used herein, the term “stalk” refers to a molecule having an amino acid sequence derived from a pIgR, but which does not comprise amino acid sequences derived from the secretory component. A stalk molecule comprises amino acid sequences that remain bound to the apical membrane following the apical proteolytic cleavage when such cleavage occurs and amino acid sequences required for such cleavage. Preferred stalk molecules confer one or more transcytotic properties to a ligand bound thereto. Most preferred are stalk molecules that confer the ability to undergo apical to basolateral transcytosis to a ligand bound thereto.

As used herein, the term “B region” refers to a non-secretory component, non-stalk region of pIgR. After transport to the apical surface of an epithelial cell, pIgR undergoes an initial cleavage, releasing a portion of pIgR into the extracellular space, with residual stalk region remaining accessable on the cell surface. The released portion undergoes further degradation by proteolytic enzymes to generate secretory component. The region that is degraded following release is referred to as the B region of pIgR. See, e.g., WO 01/72846, which is hereby incorporated by reference in its entirety. Ligands that bind to this B region can exhibit one or more transcytotic properties as described herein. B region can be an advantageous choice for directing transcytosis of a composition as described herein, as intact pIgR on a cell surface will bind to the composition without competition from secretory component.

While the preceeding is described in terms of pIgR, numerous other cell surface components provide appropriate targets for binding at the apical cell surface. The following table provides an exemplary list of such cell surface components.

| Receptor name | Type/function | Expressed in: | Ligand/substrate |
|---|---|---|---|
| Aminopeptidase N | | Coronavirus (TGEV) | Coronavirus |
| | | | (TGEV) |
| CFTR | Chloride channel; | Airway, other | Pseudomonas |
| | regulates multiple | epithelia | auriginosa LPS |
| | functions | | |
| FcRN | Rescues IgG from | Intestine, airway, | IgG (Fc) |
| | lysosomal | endothelial cells, | |
| | pathway/neonatal | placenta | |
| | IgG transport, | | |
| | intestine, placenta | | |
| CLA-1/SR-B1 | scavenger | Intestine: duodenum to | HDL, lipoproteins |
| | | rectum, liver, lung, | |
| | | ovary, adrenal glands | |
| CD-36 | scavenger | Intestine: duodenum to | Lipoproteins, |
| | | jejunum | long chain free fatty |
| | | | acids |
| ebnerin | Taste | Von Ebner's glands - | |
| | | tongue | |
| epitheliasin | Serine protease | Airway, renal tubules | |
| Anthrax protective | | Made by the bacteria | Edema factor |
| antigen | | | |
| Protective antigen | Bradley, et al. | Airway | Anthrax protective |
| receptor | Nature 2001, 414 | | antigen |
| | (6860), 225-9. | | |
| Leptin receptor | | intestine | leptin |
| Pep-T1 | Peptide transporter | intestine | peptides |
| Dipeptidylpeptidase | protease | | |
| (DPPIV) | | | |
| CD46 | | Airway (trachea) | MV (measles) |
| Sucrose-isomaltase | | intestine | apical |
| GLUT 5 | transporter | intestine | fructose |
| SGLT1 | Glucose transporter | intestine | glucose |
| Intrinsic factor | B-12 scavenger | | B-12 |
| cubilin | Non-covalent | Intestine, kidney, | B-12, intrinsic factor |
| | association with | placenta, hepatocyte, | Transferrin |
| | megalin, binds | yolk sac | |
| | intrinsic factor/B-12 | | |
| megalin | (LDL receptor | Intestine, kidney, | Cubilin-intrinsic factor- |
| | family) | placenta, hepatocyte, | B-12 |
| | | yolk sac | |
| P75NTR | Neurotrophin | | |
| | receptor | | |
| 67 kD laminin | | Intestine | |
| receptor (LR) | | | |
| EGF-R | | Intestine | |
| TGF-beta receptor I | | Intestine | |
| TGF-beta receptor | | intestine | |
| II | | | |
| Vascular | | Intestine (Caco-2) | |
| endothelial growth | | | |
| factor (VEGF) | | | |
| receptors | | | |
| Reduced folate | | intestine | |
| carrier (RFC1) | | | |
| LRP | | | Laminin/prions |
| (laminin receptor | | | |
| precursor) | | | |
| NHE 3 | Na+/H+ exchanger | intestine | |
| integrins | | | |
| ENaC | Sodium channel | Intestine | |
| 85 kD | Cryptosporidium | Caco/calf intestine | |
| CSL receptor | binds | | |
| GM1 ganglioside | | | Cholera toxin |
| GD1b | | | E. coli type II |
| | | | temperature sensitive |
| | | | enterotoxin (LTIIa) |
| BSDL | | Secreted by liver; | |
| (bile salt dependent | | Taken up by intestine | |
| lipase) | | | |
| GP 330 | Scavenger | kidney | |
| G82 antigen | | | Ab inhibits neutrophil |
| | | | migration |
| Mannose-6- | Lysosomal enzymes | Ubiquitous | |
| phosphate receptor | | | |
| CaT1 | Calcium transporter | duodenum | |
| CXCR4, CCR5 | Chemokine | intestine | Stroma cell-derived |
| | receptors | | factor 1 alpha |
| | | | Macrophage |
| | | | inflammatory protein |
| ECaC | Calcium channel | | |
| CaR | Calcium receptor | Intestine/colon | |
| HFE | Iron transport | intestine | |
| | (chelator) | | |
| GC-C | | intestine | E. coli |
| Guanylyl cyclase | | | Heat stable |
| | | | eneterotoxin (STa), |
| | | | guanylin, uroguanylin |
| IGF-II/CI-MPR | | Caco | |
| IGF-II cation | | | |
| independent | | | |
| mannose-6- | | | |
| phosphate receptor | | | |
| Cation dependent | | Caco | |
| MPR | | | |
| Sugars, | | | |
| glycocoalyx | | | |
| IGF-I, IGF-II | | intestine | |
| receptors | | | |
| A2b adenosine | | Intestine, colon, T84 | |
| receptor | | | |
| Gp330 | | intestine | |
| ((LRP relative) | | | |
| Galactosyl | | Intestine | HIV |
| ceramide | | (HT-29) | |

In addition to single-chain variable region fragments, preferred targeting moieties can be selected from the group consisting of polypeptides, recombinant polypeptides, antibodies, antibody fragments, small molecules, oligonucleotides, oligosaccharides, polysaccharides, cyclic polypeptides, peptidomimetics, and aptamers. The only limitation on such targeting moieties is that they specifically bind to a cell surface component that promotes uptake of the composition of which the targeting element is a part. As discussed above, it is particularly preferred that the targeting element bind to its cognate cell surface component at the pH present at the apical surface, but is released from the cell surface component at a pH present within the endosome.

Targeting Molecules for Uptake or Delivery at the Basolateral Cell Surface

As in the case of the apical surface, numerous molecules are known to be processed through the endocytotic/endosomal machinery of the basolateral surface. Preferred intracellular components for promoting uptake and/or delivery to the basolateral surface include, but are not limited to, receptors such as pIgR, transferrin receptor, vitamin B12 receptor, FcRn, intergrins low density lipoprotein receptor; cargo carrier fragments such as pIgR stalk, members of the PGDF, FGF, and VEGF receptor families (e.g., Flt-1, Flk-1, Flt-4, FGFR1, FGFR2, FGFR3, FGFR4). A discussion of transferrin transport follows as a general description of trafficking to the basolateral surface of polarized epithelium.

Apo-transferrin binds to iron in the blood to become holo-transferrin. At the plasma membrane, transferrin receptor binds to holo-transferrin, and the complex is internalized. Upon acidification of the endosome interior, iron dissociates for transport to the cytoplasm, while the apo-transferrin remains bound to the transferrin receptor. The receptor-apo-transferrin complex is recycled to the membrane, where the neutral pH causes dissociation of apo-transferrin back into the blood. In contrast to IgA, which undergoes basolateral-to-apical transcytosis, basolaterally internalized transferrin is efficiently recycled to the basolateral membrane.

Targeting moieties for the transferrin receptor may be generated in a variety of ways that are well known to those of skill in the art. For example, antibodies, antibody fragments, scFvs, etc., that specifically bind to the transferrin receptor may be produced by methods known in the art, such as standard immunological techniques, phage display, etc. Alternatively, transferrin (apo- or holo-) may be used as a targeting moiety. In another alternative, transferrin receptor binding peptides (“TRBP”) may be identified and used as targeting moieties. See, e.g., Eur. J. Biochem. 268: 2004-12 (2001); Campa et al., Acad. Radiol. 9: 927-32 (2002); Michon et al., Biochim. Biophys. Acta 1591: 87 (2002); Zhang et al., Sheng Wu Hua Xue Yu Sheng Wu Wu Li Xue Bao 32: 475-79 (2000). In yet another alternative, small molecules that specifically bind to transferrin receptor may be identified, e.g., from a combinatorial library. See, e.g., Benner, U.S. Pat. No. 5,958,702.

In addition to directing targeting moieties to a receptor such as the transferrin receptor, a receptor may also be indirectly targeted by providing targeting moieties that specifically bind to a ligand bound by the receptor (e.g., transferrin), but at a site that does not prevent ligand binding to the receptor. Thus, suitable targeting moieties can include those that bind to a receptor whether or not ligand is bound, those that bind to the cognate ligand whether or not ligand is bound, those that are specific for free receptor, and those that are specific for receptor bound to its cognate ligand. Preferred targeting moieties can be those targeting moieties that bind only under specific circumstances, such as those that bind to the target under conditions found in the endosome, but not under conditions found at the cell surface, or vice versa.

While the preceeding is described in terms of transferrin receptor, numerous other cellular components provide appropriate targets for delivery to the basolateral cell surface. The following table provides an exemplary list of such cellular components.

| Receptor name | Type/function | Expressed in: | Ligand/substrate |
|---|---|---|---|
| FcRN | Rescues IgG from | Intestine, airway | IgG (Fc) |
| | lysosomal | | |
| | pathway/neonatal | | |
| | IgG transport, | | |
| | intestine, placenta | | |
| TGF-beta receptor III | | intestine | |
| Transcobalamin | | Intestine, kidney, liver, | Transcobalamin- |
| receptor | | placenta | vitamin B-12 |
| Hepatocyte growth | Growth factor receptor | | |
| factor/scatter | | | |
| factor(HGF/SF) | | | |
| receptor (c-Met) | | | |
| CD4 | | Intestine | |
| Toll-like receptor 5 | | intestine | Bacterial flagellin |
| (TLR5) | | | |
| TGF-betal receptor | | Intestine | |
| ErbB receptors | | Intestine | |
| Adenosine receptor | | intestine | |
| EGF-R | | | |
| ASGP-R | | | |
| (Asialoglycoprotein | | | |
| receptor) | | | |
| LRP (low density | | intestine | |
| lipoprotein receptor | | | |
| related protein) | | | |

Just as is described above with regard to pIgR and stalk at the apical surface, a similar range of targeting moieties for binding to an appropriate cell component for uptake from and/or delivery to the basolateral surface are available for use. Thus, in addition to single-chain variable region fragments, preferred targeting moieties can be selected from the group consisting of polypeptides, recombinant polypeptides, antibodies, antibody fragments, small molecules, oligonucleotides, oligosaccharides, polysaccharides, cyclic polypeptides, peptidomimetics, and aptamers. As discussed above, it is preferred in certain embodiments that the targeting element bind to its cognate cell surface component at the pH present within the endosome, but is released from the cell surface component at a pH present at the basolateral surface; or, in the alternative, the targeting element bind to its cognate cell surface component at the pH present at the cell surface, but is released from the cell surface component at a pH present within the endosome.

Targeting Molecules for Intracellular Retention

Once the compositions of the present invention have entered cells, numerous methods known to those of skill in the art may be targeted to direct retention of the composition within an intracellular compartment. For example, the carboxy terminal tetra-peptide, KDEL, is found in many lumenal ER resident proteins. When transplanted onto various reporter molecules, it localises them to the ER showing that it is both necessary and sufficient for this process. A sorting receptor was postulated and later identified both in yeast and in mammals. The receptor, termed erd 2, localises, at steady state, to the cis side of the Golgi apparatus and redistributes upon ligand binding to the ER (Lewis and Pelham, Cell 68: 353-64 (1992). Biochemical characterisation of the receptor showed that it specifically binds the ligand and does so in a pH dependent manner with an optimum around pH 5.0. Thus, the addition of a KDEL motif to the compositions of the present invention can provide ER targeting.

Similarly, several ER resident membrane proteins have now been shown to contain signals similar to that of the KDEL motif in their cytoplasmic domains. In resident proteins with a type I topology (N-terminus is in the lumen), the signal has been shown to consist of two critical lysines which have to be in a −3 and a −4/−5 position relative to the C-terminus [-K(X)KXX] whereas in type II proteins (C-terminus is in the lumen), the signal consists of two critical arginines which have to be within the first five N-terminal residues of the protein. When transplanted onto reporter molecules, these motifs are both necessary and sufficient for ER localisation, yet allow the reporter molecule to acquire Golgi modifications. They are, therefore, similar to the KDEL motif in that they act as retrieval signals returning lost ER proteins from as far away as the trans Golgi cisternae. The K(X)KXX motif is known to bind specifically to coatomer, a component involved in vesicle mediated transport. Like the addition of a KDEL motif to the compositions of the present invention, the K(X)KXX can provide ER targeting.

Likewise, nuclear targeting is a signal-dependent, saturable process that appears to be carrier-mediated. Two types of Nuclear Localization Signal (NLS): a short basic sequences of 4-8 residues [PPKKKRKV is the NLS of SV40 large T antigen]; and a bipartite signal with two stretches of basic amino acids separated by ten less-conserved amino acids. [KRPAATKKAGQAKKKK is the NLS of nucleoplasmin]. Both types of NLS are rich in the basic amino acids arginine and lysine and usually contain proline. Importin, a component of the nuclear localization signal (NLS) receptor complex binds to the NLS of a protein to be imported. Importin-β, the other subunit of the NLS receptor complex, mediates docking with the outer surface of the nuclear pore in a rapid, energy-independent fashion. Translocation of the trimeric complex occurs in an ATP-dependent manner with importin-β interacting with the components of the pore complex. Once the complex enters the nucleoplasm, Ran (a small GTP binding protein) interacts with importin-βand mediates the dissociation of the cargo molecule from the complex by a GTP-dependent mechanism.

Peroxisomes are a family of organelles that share a common biogenetic mechanism but have different functions depending on the tissue, the developmental stage or environmental conditions. Peroxisomes are found in virtually all eukaryotes. They have a single membrane and lack DNA, so all their protein constituents are encoded by the nuclear genome and the proteins are imported from the cytoplasm. In mammals, peroxisomes have essential roles in the biosynthesis of ether-linked lipids and in the degradation of very-long-chain dicarboxylic and substituted fatty acids that are exported to the mitochondria for the final stages of degradation. Two types of peroxisomal matrix targeting signal have been characterized. The first is a C-terminal tripeptide (PTS1), the prototype for which is Ser-Lys-Leu (SKL). This signal seems to be quite degenerate in that a number of substitutions are compatible with function. However, the function of a particular sequence is dependent on both species and context. The second matrix-targeting signal (PTS2) is an N-terminal nonapeptide with the consensus sequence (R/K)(L/N/I/Q)X₅(Q/H)(L/A).

Additional motifs have been described for additional intracellular compartments. See, e.g., Shewan et al., Biochem. J. 350: 99-107 (2000) (endosome); Cherqui et al., J. Biol. Chem. 276: 13314-21 (2001) (lysosome); Honing et al., EMBO J. 15: 5230-39 (1996) (lysosome). The skilled artisan will also understand that, in addition to placing such localization signals into the compositions of the present invention, targeting could also be achieved by providing the compositions of the present invention with a targeting elemen (e.g., an antibody or sFv) that binds to endogenous proteins targeted to these compartments. For example, the composition could comprise an antibody to a LAMP, LGP, or LEP that is normally targeted to the lysosome; to a KDEL-containing protein that is normally targeted to the ER; to an endolyn that is normally targeted to the endosome; etc.

Coupling of Components

By providing a composition that comprises a plurality of targeting elements, the present invention can utilize the ability of the cell to maintain polarity of membrane domains to enhance the delivery of molecules taken up at the apical surface into the general circulation. By further associating a medically-relevant moiety with the targeting elements, the present invention can provide increased bioavailability of therapeutic, diagnostic, prophylactic, or imaging molecules.

In preferred embodiments, a molecule used to configure a particle for such delivery comprises a first element “coupled” in some sense to a second (or third, or fourth, etc.) element. The skilled artisan will understand that such elements may be simply two portions of a single molecule (an example of two such regions may be an Fc region and an Fab region on an antibody), or two molecules linked by a tethering “linker moiety.” Numerous methods are available to the skilled artisan to provide such “coupled” molecules.

For example, any two components (e.g., two components independently selected from the group consisting of a polypeptide, an antibody, an antibody fragment, a single-chain variable region fragment, a small molecule, an oligonucleotide, an oligosaccharide, a polysaccharide, a cyclic polypeptide, a peptidomimetic, and an aptamer, a poly(ethylene oxide), a dextran, etc.), may be chemically cross-linked by a linker having chemistry compatible with a site on each component. Crosslinkers are well known to those of skill in the art, and may be obtained commercially (see, e.g., Pierce Chemical Company Catalog and Handbook 1994-95, pages 0-90 through 0-110, which is hereby incorporated by reference) or synthesized as needed.

Alternatively, in cases where both components are peptides, the components may be coupled “genetically”; that is, the first and second elements may be expressed as a fusion protein. For example, U.S. Pat. No. 6,072,041 to Davis et al. is drawn to fusion proteins that are directed to the secretory component of pIgR. Ferkol et al., Am. J.Respir. Crit. Care Med. 161:944-951, 2000, discloses a fusion protein consisting of a single-chain variable region fragment directed to the secretory component (SC) of human pIgR and a human alpha (1)-antitrypsin. U.S. Pat. No. 6,042,833 to Mostov et al. discloses “genetic fusions” and “fusion proteins” that include ricin A, poly-(L)-Lys, or a phage surface protein.

In a similar manner, molecular biology may be used to introduce domains into a component that can combine with a complementary domain on a second component. For example, a coiled-coil domain sequence may be attached to a first targeting element and a second targeting element to provide the complementarity necessary to achieve binding between the two elements. Alternatively, cysteine residues may be introduced into the two targeting elements for the formation of a disulfide-bonded complex.

In an alternative approach, the various components of the compositions described herein can be associated with a particle or capsule. Methods for producing particulate administration systems for delivery of biologically-relevant molecules are well known to those of skill in the art. Such particles are preferably porous and/or biodegradable so that molecules (e.g., drugs, vaccines, vitamins, polypeptides, antibodies, etc.) contained within the particle may be released once delivered into the circulation; however, nonporous and/or nonbiodegradable particles (e.g., liposomes) are also known to those of skill in the art. Preferred particles and capsules, including microparticles, nanoparticles, microcapsules, and nanocapsules are disclosed in, e.g., U.S. Pat. No. 5,702,727; U.S. Pat. No. 5,620,708; U.S. Pat. No. 5,607,691; U.S. Pat. No. 4,610,896; U.S. Pat. No. 5,149,794; U.S. Pat. No. 6,197,349; U.S. Pat. No. 6,159,502; U.S. Pat. No. 5,785,976; Chiu et al., Biomaterials 23: 1103-12 (2002); Andrianov et al., Biomaterials 19: 109-115 (1998); Soppimath et al., J Controlled Release 70: 1-20 (2001); McPhail et al., Intl. J. Pharmaceutics 200: 73-86 (2000); Müller et al., Eur. J. Pharmaceut. Biopharmaceut. 50: 161-177 (2000); Franssen et al., J. Controlled Release 60: 211-21 (1999); Prokop et al., Biotechnol. and Bioeng. 75: 228-232 (2001); Allémann et al., Adv. Drug Deliv. Rev. 34: 171-89 (1998); Vinogradov et al., Adv. Drug Deliv. Rev. 54: 135-47 (2002); Jung et al., Eur. J. Pharmaceut. Biopharmaceut. 50: 147-60 (2000); Martin et al., Biomaterials 19: 69-76 (1998); Vervoort et al., Intl. J. Pharmaceutics 172: 137-45 (1998); J. Controlled Release 65: 49-54 (2000); Davda and Labhasetwar, Intl. J. Pharmaceutics 223: 51-9 (2002); Düzgünes and Nir, Adv. Drug Deliv. Rev. 40: 3-18 (1999); Nagayasu et al., Adv. Drug Deliv. Rev. 40: 75-87 (1999); Leroueil-Le Verger et al., Eur. J. Pharmaceut. Biopharmaceut. 46: 137-143 (1998); Breton et al., Biomaterials 19: 271-81 (1998); Konan et al., Intl. J. Pharmaceutics 233: 239-52 (2002); Duncan et al, Eur. Polymer J. 37: 1821-6 (2001); and Stenekes et al., Biomaterials 22: 1891-8 (2001), each of which is hereby incorporated by reference in its entirety. Such delivery systems may be formulated for topical, ingestible, injectable, and inhaled applications.

As discussed above, one or more molecules, e.g., targeting moieties and/or medically-relevant moieties, may be associated with particulate administration systems by numerous methods for associating such molecules with a particle or capsule are known to those of skill in the art, including covalent attachment to a component of the particle or capsule, electrostatic attachment to a component of the particle or capsule, physical entrapment of all or a portion of the molecule by the particle or capsule, and/or indirect binding to a component of the particle or capsule. See, e.g., U.S. Provisional Patent Application No. 60/402,029, entitled “COMPOSITIONS AND METHODS FOR TARGETED BIOLOGICAL DELIVERY OF MOLECULAR CARRIERS,” filed on Aug. 7, 2002 (Attorney Docket No. 2202), which is hereby incorporated by reference in its entirety.

Compositions

The compositions of the present invention provide for delivery of medically-relevant moieties, i.e., therapeutic, diagnostic, prophylactic, or imaging molecules to a subject in need thereof. The compositions of the invention can further comprise other chemical components, such as diluents and excipients. A “diluent” is a chemical compound diluted in a solvent, preferably an aqueous solvent, that facilitates dissolution of the therapeutic agent in the solvent, and it may also serve to stabilize the biologically active form of the chimeric pIgR-targeting protein or one or more of its components. Salts dissolved in buffered solutions are utilized as diluents in the art. For example, preferred diluents are buffered solutions containing one or more different salts. A preferred buffered solution is phosphate buffered saline (particularly in conjunction with compositions intended for pharmaceutical administration), as it mimics the salt conditions of human blood. Since buffer salts can control the pH of a solution at low concentrations, a buffered diluent rarely modifies the biological activity of a biologically active peptide.

An “excipient” is any more or less inert substance that can be added to a composition in order to confer a suitable property, for example, a suitable consistency or to form a drug. Suitable excipients and carriers include, in particular, fillers such as sugars, including lactose, sucrose, mannitol, or sorbitol cellulose preparations such as, for example, maize starch, wheat starch, rice starch, agar, pectin, xanthan gum, guar gum, locust bean gum, hyaluronic acid, casein potato starch, gelatin, gum tragacanth, polyacrylate, methyl cellulose, hydroxypropylmethyl-cellulose, sodium carboxymethylcellulose, and/or polyvinylpyrrolidone (PVP). If desired, disintegrating agents can also be included, such as cross-linked polyvinyl pyrrolidone, agar, or alginic acid or a salt thereof such as sodium alginate. Other suitable excipients and carriers include hydrogels, gellable hydrocolloids, and chitosan. Chitosan microspheres and microcapsules can be used as carriers. See WO 98/52547 (which describes microsphere formulations for targeting compounds to the stomach, the formulations comprising an inner core (optionally including a gelled hydrocolloid) containing one or more active ingredients, a membrane comprised of a water insoluble polymer (e.g., ethylcellulose) to control the release rate of the active ingredient(s), and an outer layer comprised of a bioadhesive cationic polymer, for example, a cationic polysaccharide, a cationic protein, and/or a synthetic cationic polymer; U.S. Pat. No. 4,895,724. Typically, chitosan is cross-linked using a suitable agent, for example, glutaraldehyde, glyoxal, epichlorohydrin, and succinaldehyde. Compositions employing chitosan as a carrier can be formulated into a variety of dosage forms, including pills, tablets, microparticles, and microspheres, including those providing for controlled release of the active ingredient(s). Other suitable bioadhesive cationic polymers include acidic gelatin, polygalactosamine, polyamino acids such as polylysine, polyhistidine, polyornithine, polyquaternary compounds, prolamine, polyimine, diethylaminoethyldextran (DEAE), DEAE-imine, DEAE-methacrylate, DEAE-acrylamide, DEAE-dextran, DEAE-cellulose, poly-p-aminostyrene, polyoxethane, copolymethacrylates, polyamidoamines, cationic starches, polyvinylpyridine, and polythiodiethylaminomethylethylene.

The compositions of the invention can be formulated in any suitable manner. Suitable formulations include dry and liquid formulations. Dry formulations include freeze dried and lyophilized powders, which are particularly well suited for aerosol delivery to the sinuses or lung, or for long term storage followed by reconstitution in a suitable diluent prior to administration. Other preferred dry formulations include those wherein a composition according to the invention is compressed into tablet or pill form suitable for oral administration or compounded into a sustained release formulation. As those in the art will appreciate, the compositions of the invention can be placed into any suitable dosage form. Pills and tablets represent some of such dosage forms. The compositions can also be encapsulated into any suitable capsule or other coating material, for example, by compression, dipping, pan coating, spray drying, etc. Suitable capsules include those made from gelatin and starch. In turn, such capsules can be coated with one or more additional materials, for example, and enteric coating, if desired. Liquid formulations include aqueous formulations, gels, and emulsions.

Some preferred embodiments concern compositions that comprise a bioadhesive, preferably a mucoadhesive, coating. A “bioadhesive coating” is a coating that allows a substance (e.g., a according to the invention) to adhere to a biological surface or substance better than occurs absent the coating. A “mucoadhesive coating” is a preferred bioadhesive coating that allows a substance, for example, a composition according to the invention, to adhere better to mucosa than would occur absent the coating. For example, micronized particles (e.g., particles having a mean diameter of about 5, 10, 25, 50, or 100 μm) can be coated with a mucoadhesive. The coated particles can then be assembled into a dosage form suitable for delivery to an organism. Preferably, and depending upon the location where the cell surface transport moiety to be targeted is expressed, the dosage form is then coated with another coating to protect the formulation until it reaches the desired location, where the mucoadhesive enables the formulation to be retained while the chimeric pIgR-targeting proteins interact with the target cell surface transport moiety.

The particular amount of biologically active component to be delivered will depend on many factors, including the effect to be achieved, the type of organism to which the composition is delivered, delivery route, dosage regimen, and the age, health, and sex of the organism. As such, the particular dosage is left to the ordinarily skilled artisan's discretion.

Depending on the mode of delivery employed, the context-dependent functional entity can be delivered in a variety of pharmaceutically acceptable forms. For example, the context-dependent functional entity can be delivered in the form of a solid, solution, emulsion, dispersion, micelle, liposome, and the like, incorporated into a pill, capsule, tablet, suppository, aerosol, droplet, or spray. Pills, tablets, suppositories, aerosols, powders, droplets, and sprays may have complex, multilayer structures and have a large range of sizes. Aerosols, powders, droplets, and sprays may range from small (1 micron) to large (200 micron) in size.

The compositions of the present invention can comprise the usual non-toxic, pharmaceutically acceptable carriers for tablets, pellets, capsules, suppositories, solutions, emulsions, suspensions, and any other form suitable for use. The carriers that can be used include glucose, lactose, mannose, sucrose, gum acacia, gelatin, mannitol, starch paste, magnesium trisilicate, talc, cornstarch, keratin, colloidal silica, potato starch, urea, medium chain length triglycerides, dextrans, and other carriers suitable for use in manufacturing preparations, in solid, semisolid, or liquid form. In addition auxiliary, stabilizing, thickening and coloring agents and perfumes may be used. Examples of a stabilizing dry agent includes triulose and or trehalose, preferably at concentrations of 0.1% or greater (See, e.g., U.S. Pat. No. 5,314,695).

Pharmaceutical formulations of particular interest in the context of the invention include, but are not limited to, those taught in U.S. Pat. No. 5,254,342, entitled “Compositions and methods for enhanced transepithelial and transendothelial transport o[f] active agents” to Shen et al.; and U.S. Pat. No. 6,110,456, “Oral delivery o[f] adeno-associated viral vectors.”

The compositions of the present invention may be used in therapeutic, prophylactic, diagnostic, and/or imaging methods. The compositions of the present invention may also be used with neutraceuticals and other nutritional and dietary supplements.

Nucleic acids for use as medically-relevant moieties in the present invention include, but are not limited to, catalytic nucleic acids, e.g., ribozymes; structural nucleic acids, e.g., ribosomal RNA (rRNA); transfer RNA (tRNA); antisense nucleic acids, e.g., antisense oligonucleotides; aptamers; nucleic acids decoys, e.g., dsDNA comprising sequences to which DNA-binding proteins specifically bind; and expression elements that direct the in vivo production of a biologically active nucleic acid or polypeptide. The biologically active nucleic acids of the invention may be of any shape, form or topology including, but not limited to, double-stranded (ds), including A-, B- and Z-DNA; single-stranded (ss); mixed ds and ss; linear; circular; hybrid (e.g., RNA:DNA hybrids); supercoiled; compacted; nicked; complexed with other nucleic acids and/or polypeptides; etc.

Polypeptides for use as medically-relevant moieties in the present invention include, but are not limited to, (1) antibodies or fragments thereof; (2) endogenous polypeptides (e.g., enzymes, cytokines, polypeptide hormones) that are missing, deficient, mutated or underexpressed in a patient suffering from a particular disease or disorder; (3) endogenous polypeptides (e.g., enzymes, cytokines, polypeptide hormones) that can be overexpressed to achieve a biological effect; (4) exogenous proteins such as recombinant peptides; (5) enzymes for use in ADEPT methods, etc. Some therapies involving polypeptides of type (2) are designed so as to be therapeutic for inborn errors of metabolism and include, by way of non-limiting example, enzyme replacement (e.g., Factor IX in the case of hemophilia B, and phenylalanine hydroxylase in the case of phenylketonuria) therapy, and protein or factor replacement (e.g., Factor VIII in the case of hemophilia A, and insulin in the case of Type I diabetes) therapy. See, e.g., Dai et al., Proc. Natl. Acad. Sci. USA 89:10892-10895, 1992; and Wang et al., Proc. Natl. Acad. Sci. USA 96:3906-3910, 1999.

An additional exemplary list of suitable compounds for use as medically-relevant moieties in the present invention is provided below:

analgesics/antipyretics (e.g., aspirin, acetaminophen, ibuprofen, naproxen sodium, buprenorphine hydrochloride, propoxyphene hydrochloride, propoxyphene napsylate, meperidine hydrochloride, hydromorphone hydrochloride, morphine sulfate, oxycodone hydrochloride, codeine phosphate, dihydrocodeine bitartrate, pentazocine hydrochloride, hydrocodone bitartrate, levorphanol tartrate, diflunisal, trolamine salicylate, nalbuphine hydrochloride, mefenamic acid, butorphanol tartrate, choline salicylate, butalbital, phenyltoloxamine citrate, diphenhydramine citrate, methotrimeprazine, cinnamedrine hydrochloride, meprobamate, and the like);

antimigraine agents (e.g., ergotamine tartrate, propanolol hydrochloride, isometheptene mucate, dichloralphenazone, and the like);

sedatives/hypnotics (e.g., barbiturates (e.g., pentobarbital, pentobarbital sodium, secobarbital sodium), benzodiazapines (e.g., flurazepam hydrochloride, triazolam, tomazeparm, midazolam hydrochloride, and the like);

antianginal agents (e.g., beta-adrenergic blockers, calcium channel blockers (e.g., nifedipine, diltiazem hydrochloride, and the like), nitrates (e.g., nitroglycerin, isosorbide dinitrate, pentaerythritol tetranitrate, erythrityl tetranitrate, and the like));

antianxiety agents (e.g., lorazepam, buspirone hydrochloride, prazepam, chlordiazepoxide hydrochloride, oxazepam, clorazepate dipotassium, diazepam, hydroxyzine pamoate, hydroxyzine hydrochloride, alprazolam, droperidol, halazepam, chlormezanone, and the like);

antipsychotic agents (e.g., haloperidol, loxapine succinate, loxapine hydrochloride, thioridazine, thioridazine hydrochloride, thiothixene, fluphenazine hydrochloride, fluphenazine decanoate, fluphenazine enanthate, trifluoperazine hydrochloride, chlorpromazine hydrochloride, perphenazine, lithium citrate, prochlorperazine, and the like); antimanic agents (e.g., lithium carbonate);

antiarrhythmics (e.g., bretylium tosylate, esmolol hydrochloride, verapamil hydrochloride, amiodarone, encainide hydrochloride, digoxin, digitoxin, mexiletine hydrochloride, disopyramide phosphate, procainamide hydrochloride, quinidine sulfate, quinidine gluconate, quinidine polygalacturonate, flecainide acetate, tocainide hydrochloride, lidocaine hydrochloride, and the like);

antiarthritic agents (e.g., phenylbutazone, sulindac, penicillamine, salsalate, piroxicam, azathioprine, indomethacin, meclofenamate sodium, gold sodium thiomalate, ketoprofen, auranofin, aurothioglucose, tolmetin sodium, and the like);

antigout agents (e.g., colchicine, allopurinol, and the like);

anticoagulants (e.g., heparin (a repeating disaccharide unit of D-glucosamine and uronic acid linked by 1→4 interglycosidic bond having a molecular weight of between about 6000 to about 40000 daltons, usually between 12000 and 15000 daltons), heparin sodium, warfarin sodium, and the like);

thrombolytic agents (e.g., urokinase, streptokinase, altoplase, and the like);

antifibrinolytic agents (e.g., aminocaproic acid);

hemorheologic agents (e.g., pentoxifylline);

antiplatelet agents (e.g., aspirin, empirin, ascriptin, and the like);

anticonvulsants (e.g., valproic acid, divalproate sodium, phenyloin, phenyloin sodium, clonazepam, primidone, phenobarbitol, phenobarbitol sodium, carbamazepine, amobarbital sodium, methsuximide, metharbital, mephobarbital, mephenyloin, phensuximide, paramethadione, ethotoin, phenacemide, secobarbitol sodium, clorazepate dipotassium, trimethadione, and the like);

antiparkinson agents (e.g., ethosuximide, and the like); antidepressants (e.g., doxepin hydrochloride, amoxapine, trazodone hydrochloride, amitriptyline hydrochloride, maprotiline hydrochloride, phenelzine sulfate, desipramine hydrochloride, nortriptyline hydrochloride, tranylcypromine sulfate, fluoxetine hydrochloride, doxepin hydrochloride, imipramine hydrochloride, imipramine pamoate, nortriptyline, amitriptyline hydrochloride, isocarboxazid, desipramine hydrochloride, trimipramine maleate, protriptyline hydrochloride, and the like);

antihistamines/antipruritics (e.g., hydroxyzine hydrochloride, diphenhydramine hydrochloride, chlorpheniramine maleate, brompheniramine maleate, cyproheptadine hydrochloride, terfenadine, clemastine fumarate, triprolidine hydrochloride, carbinoxamine maleate, diphenylpyraline hydrochloride, phenindamine tartrate, azatadine maleate, tripelennamine hydrochloride, dexchlorpheniramine maleate, methdilazine hydrochloride, trimprazine tartrate and the like);

antihypertensive agents (e.g., trimethaphan camsylate, phenoxybenzamine hydrochloride, pargyline hydrochloride, deserpidine, diazoxide, guanethidine monosulfate, minoxidil, rescinnamine, sodium nitroprusside, rauwolfia serpentina, alseroxylon, phentolamine mesylate, reserpine, and the like);

agents useful for calcium regulation (e.g., calcitonin, parathyroid hormone, and the like);

antibacterial agents (e.g., amikacin sulfate, aztreonam, chloramphenicol, chloramphenicol palmitate, chloramphenicol sodium succinate, ciprofloxacin hydrochloride, clindamycin hydrochloride, clindamycin palmitate, clindamycin phosphate, metronidazole, metronidazole hydrochloride, gentamicin sulfate, lincomycin hydrochloride, tobramycin sulfate, vancomycin hydrochloride, polymyxin B sulfate, colistimethate sodium, colistin sulfate, and the like);

antifungal agents (e.g., griseofulvin, keloconazole, and the like);

antiviral agents (e.g., interferon gamma, zidovudine, amantadine hydrochloride, ribavirin, acyclovir, and the like);

antimicrobials (e.g., cephalosporins (e.g., cefazolin sodium, cephradine, cefaclor, cephapirin sodium, ceftizoxime sodium, cefoperazone sodium, cefotetan disodium, cefutoxime azotil, cefotaxime sodium, cefadroxil monohydrate, ceftazidime, cephalexin, cephalothin sodium, cephalexin hydrochloride monohydrate, cefamandole nafate, cefoxitin sodium, cefonicid sodium, ceforanide, ceftriaxone sodium, ceftazidime, cefadroxil, cephradine, cefuroxime sodium, and the like), penicillins (e.g., ampicillin, amoxicillin, penicillin G benzathine, cyclacillin, ampicillin sodium, penicillin G potassium, penicillin V potassium, piperacillin sodium, oxacillin sodium, bacampicillin hydrochloride, cloxacillin sodium, ticarcillin disodium, azlocillin sodium, carbenicillin indanyl sodium, penicillin G potassium, penicillin G procaine, methicillin sodium, nafcillin sodium, and the like), erythromycins (e.g., erythromycin ethylsuccinate, erythromycin, erythromycin estolate, erythromycin lactobionate, erythromycin siearate, erythromycin ethylsuccinate, and the like), tetracyclines (e.g., tetracycline hydrochloride, doxycycline hyclate, minocycline hydrochloride, and the like), and the like);

anti-infectives (e.g., GM-CSF);

bronchodialators (e.g., sympathomimetics (e.g., epinephrine hydrochloride, metaproterenol sulfate, terbutaline sulfate, isoetharine, isoetharine mesylate, isoetharine hydrochloride, albuterol sulfate, albuterol, bitolterol, mesylate isoproterenol hydrochloride, terbutaline sulfate, epinephrine bitartrate, metaproterenol sulfate, epinephrine, epinephrine bitartrate), anticholinergic agents (e.g., ipratropium bromide), xanthines (e.g., aminophylline, dyphylline, metaproterenol sulfate, aminophylline), mast cell stabilizers (e.g., cromolyn sodium), inhalant corticosteroids (e.g., flurisolidebeclomethasone dipropionate, beclomethasone dipropionate monohydrate), salbutamol, beclomethasone dipropionate (BDP), ipratropium bromide, budesonide, ketotifen, salmeterol, xinafoate, terbutaline sulfate, triamcinolone, theophylline, nedocromil sodium, metaproterenol sulfate, albuterol, flunisolide, and the like);

cytokines (e.g., interleukins IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, interferons alpha, beta, and gamma;

growth factors (e.g., growth hormone, insulin-like growth factor 1 and 2 (IGF-1 and IGF-2), vascular endothelial growth factor (VEGF), granulocyte colony stimulating factor (GCSF), glucose sensitive factor (GSF), platelet derived growth factor (PGDF), epidermal growth factor (EGF), fibroblast growth factor (FGF), transforming growth factor beta (TGF-beta));

tumor necrosis factor (TNF), TNF receptor, TNF inhibitors (e.g., etanercept and infliximab), and antibodies to TNF or its receptor;

hormones (e.g., androgens (e.g., danazol, testosterone cypionate, fluoxymesterone, ethyltostosterone, testosterone enanihate, methyltestosterone, fluoxymesterone, testosterone cypionate), estrogens (e.g., estradiol, estropipate, conjugated estrogens), progestins (e.g., methoxyprogesterone acetate, norethindrone acetate), corticosteroids (e.g., triamcinolone, betamethasone, betamethasone sodium phosphate, dexamethasone, dexamethasone sodium phosphate, dexamethasone acetate, prednisone, methylprednisolone acetate suspension, triamcinolone acetonide, methylprednisolone, prednisolone sodium phosphate methylprednisolone sodium succinate, hydrocortisone sodium succinate, methylprednisolone sodium succinate, triamcinolone hexacatonide, hydrocortisone, hydrocortisone cypionate, prednisolone, fluorocortisone acetate, paramethasone acetate, prednisolone tebulate, prednisolone acetate, prednisolone sodium phosphate, hydrocortisone sodium succinate, and the like), thyroid hormones (e.g., levothyroxine sodium) and the like), erythropoietin (EPO), and the like;

hypoglycemic agents (e.g., purified or recombinant human insulin, purified or recombinant beef insulin, purified or recombinant pork insulin, glyburide, chlorpropamide, glipizide, tolbutamide, tolazamide, and the like);

hypolipidemic agents (e.g., clofibrate, dextrothyroxine sodium, probucol, lovastatin, niacin, and the like);

proteins (e.g., DNase, alginase, superoxide dismutase, lipase, antibodies, and the like, synthetic proteins, recombinant proteins, chimeric proteins (i.e., comprising domains derived from more than one protein));

nucleic acids (e.g., sense or anti-sense nucleic acids encoding any protein suitable for delivery by inhalation, including the proteins described herein, and the like);

agents useful for erythropoiesis stimulation (e.g., erythropoietin);

antiulcer/antireflux agents (e.g., famotidine, cimetidine, ranitidine hydrochloride, and the like); and

antinauseants/antiemetics (e.g., meclizine hydrochloride, nabilone, prochlorperazine, dimenhydrinate, promethazine hydrochloride, thiethylperazine, scopolamine, and the like).

This list is not intended to be limiting. Additional agents contemplated for delivery employing the devices and methods described herein include agents useful for the treatment of diabetes (e.g., activin, glucagon, insulin, somatostatin, proinsulin, amylin, and the like), carcinomas (e.g., taxol, interleukin-1, interleukin-2 (especially useful for treatment of renal carcinoma), and the like, as well as leuprolide acetate, LHRH analogs (such as nafarelin acetate), and the like, which are especially useful for the treatment of prostatic carcinoma), endometriosis (e.g., LHRH analogs), uterine contraction (e.g., oxytocin), diuresis (e.g., vasopressin), cystic fibrosis (e.g., DNase (i.e., deoxyribonuclease), SLPI, and the like), neutropenia (e.g., GCSF), MS (e.g., beta 1-interferon), respiratory disorders (e.g., superoxide dismutase), RDS (e.g., surfactants, optionally including apoproteins), obesity (e.g., leptins) and the like.

Similarly, functional derivatives of the foregoing agents are also within the scope of the present invention. The term “functional derivative” indicates a chemically modified version, an analog, or a homolog of a compound that retains a biological function of interest of that compound for any given application. In the case of polypeptides, chemical modification may include, by way of non-limiting example, adding chemical groups to a compound (e.g., glycosylation, phosphorylation, thiolation, pegylation, etc.), eliminating parts of a compound that do not impact the function of interest (preparing a truncated form of a protein that retains an activity of interest, e.g., Klenow fragment), changing sets of one or more amino acids in the polypeptide (preparing muteins); analogs are exemplified by peptidomimetics; and homologs are polypeptides from other species of animals that retain biological activity (e.g., human and porcine insulin, human and salmon calcitonin, etc.) or intraspecies isomers of a polypeptide (protein “families” such as the cytochrome P450 family).

It will be readily apparent to those skilled in the relevant arts that other suitable modifications and adaptations to the methods and applications described herein may be made without departing from the scope of the invention or any embodiment thereof. Having now described the present invention in detail, the same will be more clearly understood by reference to the following examples, which are included herewith for purposes of illustration only and are not intended to be limiting of the invention.

Preparation of Particles and Capsules

Methods for producing particulate administration systems for delivery of medically-relevant molecules are well known to those of skill in the art. Such particles are preferably porous and/or biodegradable so that molecules (e.g., drugs, vaccines, vitamins, polypeptides, antibodies, etc.) contained within the particle may be released once delivered into the circulation; however, nonporous and/or nonbiodegradable particles (e.g., liposomes) are also known to those of skill in the art. Preferred particles and capsules, including microparticles, nanoparticles, microcapsules, and nanocapsules are disclosed in, e.g., U.S. Pat. No. 5,702,727; U.S. Pat. No. 5,620,708; U.S. Pat. No. 5,607,691; U.S. Pat. No. 4,610,896; U.S. Pat. No. 5,149,794; U.S. Pat. No. 6,197,349; U.S. Pat. No. 6,159,502; U.S. Pat. No. 5,785,976; Chiu et al., Biomaterials 23: 1103-12 (2002); Andrianov et al., Biomaterials 19: 109-115 (1998); Soppimath et al., J. Controlled Release 70: 1-20 (2001); McPhail et al., Intl. J. Pharmaceutics 200: 73-86 (2000); Müller et al., Eur. J. Pharmaceut. Biopharmaceut. 50: 161-177 (2000); Franssen et al., J. Controlled Release 60: 211-21 (1999); Prokop et al., Biotechnol. and Bioeng. 75: 228-232 (2001); Allémann et al., Adv. Drug Deliv. Rev. 34: 171-89 (1998); Vinogradov et al., Adv. Drug Deliv. Rev. 54: 135-47 (2002); Jung et al., Eur. J. Pharmaceut. Biopharmaceut. 50: 147-60 (2000); Martin et al., Biomaterials 19: 69-76 (1998); Vervoort et al., Intl. J. Pharmaceutics 172: 137-45 (1998); J. Controlled Release 65: 49-54 (2000); Davda and Labhasetwar, Intl. J. Pharmaceutics 223: 51-9 (2002); Düzgünes and Nir, Adv. Drug Deliv. Rev. 40: 3-18 (1999); Nagayasu et al., Adv. Drug Deliv. Rev. 40: 75-87 (1999); Leroueil-Le Verger et al., Eur. J. Pharmaceut. Biopharmaceut. 46: 137-143 (1998); Breton et al., Biomaterials 19: 271-81 (1998); Konan et al., Intl. J. Pharmaceutics 233: 239-52 (2002); Duncan et al, Eur. Polymer J. 37: 1821-6 (2001); and Stenekes et al., Biomaterials 22: 1891-8 (2001), each of which is hereby incorporated by reference in its entirety. Such delivery systems may be formulated for topical, ingestible, injectable, and inhaled applications.

It will be readily apparent to those skilled in the relevant arts that other suitable modifications and adaptations to the methods and applications described herein may be made without departing from the scope of the invention or any embodiment thereof. Having now described the present invention in detail, the same will be more clearly understood by reference to the following examples, which are included herewith for purposes of illustration only and are not intended to be limiting of the invention.

### EXAMPLES

### Example 1

### Preparation of an scFv-transferrin Bispecific Ligand

A pIgR-specific scFv, 5AFcys (see, e.g., International Publication No. WO02/28408), was coupled to human transferrin using the non-reducible heterobifunctional crosslinker LC-SMCC (Pierce). To activate the thiol group of the cysteine residue located near the C-terminus of 5AFcys, the scFv was reduced with 10 mM dithiothreitol (DTT) for 30 minutes at room temperature. The reduced ligand was run on a Superdex 75 FPLC column equilibrated in PE pH 6.25 (10 mM sodium phosphate pH 6.25, 100 mM NaCl, 1 mM EDTA). Three major elution peaks were seen. The first contained 5AFcys dimer, the second peak contained 5AFcys monomer and the last peak contained DTT. Fractions containing monomer and dimer were pooled separately and stored on ice until needed.

Human fransferrin was dissolved in PE buffer, pH 7.25, to a concentration of 10 mg/ml. A 2.5-fold molar excess of the non-reducible crosslinker LC-SMCC was dissolved in DMSO and added to the transferrin with constant stirring for 1 h at room temperature. The excess crosslinker was separated from the derivatized transferrin on a G-25 sepharose column equilibrated in PE buffer, pH 7.25.

A 5-fold molar excess of 5AFcys monomer was added to derivatized transferrin and the mixture was placed on a rotating platform overnight at 4° C. For 5AFcys dimer, a 3-fold molar excess was used. The 5AFcys-human Tf conjugate was separated from unconjugated 5AFcys on a Superdex-75 column equilibrated in 50 mM HEPES pH 7.5, 5% sucrose and 100 mM NaCl. Column fractions were run on a 4-15% SDS-PAGE gel and stained with Coomassie blue. Fractions containing the 5AFcys-human Tf conjugates were pooled and stored frozen.

### Example 2

### Preparation of an scFv-TRBP Bispecific Ligand

Eur. J. Biochem. 268: 2004-2012 (2001) discloses two peptides that were found to bind specifically to the human transferrin receptor without inhibiting transferrin binding. These two peptides, with the sequences HAIYPRH and THRPPMWSPVWP, were identified through several rounds of positive and negative selection of a phage display peptide library. When expressed at the C-terminus of a GFP fusion protein, both transferrin receptor binding peptides (TRBP) colocalized with the human transferrin receptor within endosomal compartments; also, the binding affinity of the THRPPMWSPVWP peptide was calculated to be 1.5×10-8 M, which is similar to the binding affinity of transferrin.

To create a bispecific ligand consisting of an scFv specific to pIgR and the THRPPMWSPVWP peptide, a genetic fusion was constructed in which a TRBP encoding sequence was inserted between the sequences encoding the pel-B leader and the beginning of the scFv encoding sequence using the methods described in FIGS. 1-3. Using an scFv in the vector pSyn as a DNA template, PCR was used to create a DNA fragment encoding the pel-B leader and TRBP peptide, flanked by a Hind III site at the 5′ end (in sequences contained in the pSyn plasmid vector), and Xma I and Pst I sites at the 3′ end. A Kpn I site was incorporated at the 5′ end of the TRBP encoding sequence. These sites allow one to replace the TRBP sequence or add a spacer region between TRBP and the scFv. The creation of these sites resulted in the addition of a glycine residue on the N-terminus and glycine-leucine residues to the C-terminus of the TRBP. An anti-sense PCR oligo (oligo “B”) with the sequence 5′-CAC-CTG-CAG-CCC-GGG-CCA-CAC-CGG-GCT-CCA-CAT-CGG-CGG-GCG-GTG-GGT-ACC-GGC-CA T-GGC-CGG-CTG-GGC-3′, is complementary to the minus strand of the 3′ end of the pel-B leader, and adds the Kpn I site, TRBP, the Pst I and Xma I sites to the 3′ end of the Pelb leader. A sense PCR oligo (oligo “C”) with the sequence 5′-GTG-GCC-CGG-GCT-GCA-GGT-GCA-GCT-GGT-GCA-ATC-3′, was used to add the Xma I and Pst I sites to the 5′ end of the scFv heavy chain. An oligo (LMB3) (oligo “A”), which is complementary to sequences in the vector pSyn, was paired with oligo “B” for PCR, and an scFv specific oligo (oligo “D”), was paired with oligo “C”. The PCR fragment containing the Pelb leader and TRBP was digested with Hind III and Xma I, while the 5A heavy chain PCR fragment was digested with Xma I and BamHI. The fragments were ligated together and substituted into the pSyn scFv plasmid construct to make a full-length genetic fusion protein.

This construct was expressed in E. coli and purified protein was isolated by FPLC using a Protein-A affinity column followed by purification on an immobilized metal affinity column.

### Example 3

### Transwell Transcytosis Assay

The transcytosis efficiency of the bispecific or multispecific ligands can be determined using polarized cells, such as Madin-Darby Canine Kidney cells. See, e.g., Brown et al., Traffic 1: 124-40 (2000). Other appropriate cells for use in transcytosis assays include primary cultures of polarized epithelial or endothelial cells; CaLu-3; Caco-2; EC219; WIF-B; HEP-G2; IRPT (immortalized rat proximal tubule); Int-407; BEAS-2B; Detroit 562; LLC-PK; OK; BeWo; FRT (Fischer rat thyroid); RPEJ (immortalized retinal pigment epithelial); HT29; or other appropriate cells that preferably form polarized cell layers in suitable culture systems. May be transfected if necessary to express appropriate targets for binding of the bispecific or multispecific ligands described herein.

MDCK cells expressing pIgR and the human transferrin receptor were grown in Transwell® permeable tissue culture supports (Costar), which allows the cells to receive nutrients from the top and bottom sides of the cell monolayer. Each permeable well of a 12-well Transwell® plate was seeded with 5×10⁵ cells and grown for 3 to 5 days. When the MDCK cell layer becomes confluent, the cells are oriented with their apical membrane facing upwards. Tight junctions form between the cells to prevent paracellular movement of proteins.

Bispecific ligand was added to the apical side (2 μg in 300 μl media) of the Transwell® cup while the basolateral chamber contained 800 μl media. The plate was placed in a 37° C. incubator for 16 h. The apical and basolateral media was transferred to microfuge tubes and the cell layers were washed three times with cold PBS (10 mM sodium phosphate pH 7.3, 150 mM NaCl), then lysed with 250 μl 1% NP-40 in PBS. The cell lysates were transferred to microfuge tubes and centrifuged for 5 minutes at 16,000×g to pellet the nuclei. The soluble lysates were transferred to new tubes and 100 μl of 10% Protein A-sepharose beads was added to each apical, basolateral and cell lysate tube. The tubes were placed on a rotating platform overnight at 4° C. to allow the scFv portion of the ligand to bind to protein A.

After washing the protein A-sepharose beads three times with PBS, 100 μl of non-reducing sample buffer was added to each tube and heated at 90° C. for 3 minutes. The samples were run on 4-15% SDS-PAGE gels and then transferred to PVDF membranes. Western blot analysis was done on the PVDF membranes by probing with a rabbit antibody specific to the 5A ligand. A donkey anti-rabbit antibody conjugated to alkaline phosphatase was used as the secondary antibody. The bands were detected using bromo-chloro-indolyl phosphate (BCIP) and Nitro-blue tetrazolium (NBT).

Using such an assay to examine transcytosis of 5AFcys-Tf conjugate (Example 1), it was determined that about 40% of added bispecific ligand was recoverable from the apical medium, and about 38% from the basolateral medium, with the remaining 22% being associated with the cells. Addition of excess transferrin to the basolateral medium of the transwell assay saturated the transferrin binding site, and reduced the amount of bispecific ligand present in the basolateral medium to about 32%, indicating that the transferrin receptor is utilized for transcytosis.

The contents of the articles, patents, and patent applications, and all other documents and electronically available information mentioned or cited herein, are hereby incorporated by reference in their entirety to the same extent as if each individual publication was specifically and individually indicated to be incorporated by reference. Applicants reserve the right to physically incorporate into this application any and all materials and information from any such articles, patents, patent applications, or other documents.

The inventions illustratively described herein may suitably be practiced in the absence of any element or elements, limitation or limitations, not specifically disclosed herein. Thus, for example, the terms “comprising”, “including,” “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims. In addition, where features or aspects of the invention are described in terms of Markush groups, those skilled in the art will recognize that the invention is also thereby described in terms of any individual member or subgroup of members of the Markush group.

We claim:

1. A composition comprising:
a first targeting element that specifically binds to a first cell surface component, and a second targeting element that specifically binds to a second component of the cell,
wherein said first and second targeting elements promote delivery of all or a portion of the composition into or across a layer of polarized cells comprising said first cell surface component and said second component in comparison to compositions lacking one or both of said first and second targeting elements.

2. A composition according to claim 1, wherein said first targeting element specifically binds to component present at the apical surface of said polarized cells that promotes active transport, endocytosis or transcytosis, and said second targeting element specifically binds to an intracellular component that promotes delivery to the basolateral surface of said polarized cells.

3. A composition according to claim 1, wherein said first targeting element specifically binds to component present at the basolateral surface of said polarized cells that promotes active transport, endocytosis or transcytosis, and said second targeting element specifically binds to an intracellular component that promotes delivery to the apical surface of said polarized cells.

4. A composition according to claim 1, wherein said first targeting element specifically binds to component present at the apical or basolateral surface of said polarized cells that promotes active transport, endocytosis or transcytosis, and said second targeting element specifically binds to an intracellular component that promotes retention of all or a portion of the composition within said cells.

5. A composition according to claim 1, wherein said first targeting element specifically binds to component present in a mucus layer at a surface of said polarized cells, and said second targeting element specifically binds to a component present at said surface that promotes active transport, endocytosis or transcytosis to into or across said polarized cells.

6. A composition according to claim 1, wherein the ability of said first and second targeting elements to promote delivery of all or a portion of the composition to into or across a layer of said polarized cells is determined in an in vitro transcytosis assay.

7. A composition according to claim 6, wherein said polarized cells are cultured Madin-Darby Canine Kidney cells expressing said first cell surface component and said second component.

8. A composition according to claim 6, wherein said polarized cells are cultured Caco-2 cells expressing said first cell surface component and said second component.

9. A composition according to claim 6, wherein said polarized cells are cultured CaLu-3 cells expressing said first cell surface component and said second component.

10. A composition according to claim 6, wherein said polarized cells are cultured HT29 cells expressing said first cell surface component and said second component.

11. A composition according to claim 1, wherein one or both of said first cell surface component and said second component are expressed in said polarized cells by transfecting an exogenous nucelic acid encoding one or both components into said polarized cells.

12. A composition according to claim 1, further comprising a medically-relevant moiety.

13. A composition according to claim 12, wherein said medically-relevant moiety is a therapeutic moiety.

14. A composition according to claim 1, wherein said first targeting element binds to pIgR.

15. A composition according to claim 1, wherein said first targeting element binds to a non-secretory component region of pIgR.

16. A composition according to claim 1, wherein said first targeting element binds to B region of pIgR

17. A composition according to claim 1, wherein said first targeting element binds to pIgR stalk.

18. A composition according to claim 1, wherein said first targeting element is selected from the group consisting of a polypeptide, a recombinant polypeptide, an antibody, an antibody fragment, a single-chain variable region fragment, a small molecule, an oligonucleotide, an oligosaccharide, a polysaccharide, a cyclic polypeptide, a peptidomimetic, and an aptamer.

19. A composition according to claim 1, wherein said cell surface component is selected from the group consisting of pIgR, pIgR stalk, transferrin receptor, apo-transferrin, holo-transferrin, vitamin B12 receptor, FcRn, an integrin, Flt-1, Flk-1, Flt-4, a GPI-linked protein, a scavenger receptor, and low density lipoprotein receptor.

20. A composition according to claim 1, wherein said second targeting element is selected from the group consisting of a polypeptide, a recombinant polypeptide, an antibody, an antibody fragment, a single-chain variable region fragment, a small molecule, an oligonucleotide, an oligosaccharide, a polysaccharide, a cyclic polypeptide, a peptidomimetic, and an aptamer.

21. A composition according to claim 1, wherein said second component is selected from the group consisting of pIgR, pIgR stalk, transferrin receptor, apo-transferrin, holo-transferrin, vitamin B12 receptor, FcRn, an integrin, Flt-1, Flk-1, Flt-4, a GPI-linked protein, a scavenger receptor, and low density lipoprotein receptor.

22. A composition according to claim 13, wherein said therapeutic moiety is selected from the group consisting of a polypeptide, a recombinant polypeptide, an antibody, an antibody fragment, a single-chain variable region fragment, a small molecule, an oligonucleotide, an oligosaccharide, a polysaccharide, a cyclic polypeptide, a peptidomimetic, and an aptamer.

23. A composition according to claim 22, wherein said medically-relevant moiety is an antibody, and said antibody is covalently or noncovalently bound to one or more first targeting moieties that are single-chain variable region fragments that specifically bind to pIgR.

24. A composition according to claim 18, wherein the first targeting element is an antibody or fragment thereof or a single-chain variable region fragment, and said first targeting element is covalently or noncovalently bound to a second targeting element selected from the group consisting of transferrin, antibody to transferrin, antibody to transferrin receptor, and transferrin binding peptide.

25. A composition according to claim 1, wherein said composition comprises a particulate structure selected from the group consisting of a nanoparticle, a microparticle, a nanocapsule, and a microcapsule.

26. A method of preparing a composition, comprising:
associating a first targeting element that specifically binds to a first cell surface component, and a second targeting element that specifically binds to a second component of the cell with a medically-relevant moiety,
wherein said first and second targeting elements promote delivery of all or a portion of the composition to, into, or across a layer of epithelial cells comprising said first cell surface component and said second component, in comparison to compositions lacking one or both of said first and second targeting elements.

27. A composition according to claim 26, wherein said medically-relevant moiety is a therapeutic moiety.

28. A method according to claim 26, wherein said first targeting element and said second targeting element are covalently or noncovalently bound to one another.

29. A method according to claim 26, wherein said first targeting element and said medically-relevant moiety are covalently or noncovalently bound to one another.

30. A method according to claim 26, wherein said second targeting element and said medically-relevant moiety are covalently or noncovalently bound to one another.

31. A method according to claim 26, wherein said first and second targeting elements are bound to a particulate structure selected from the group consisting of a nanoparticle, a microparticle, a nanocapsule, and a microcapsule.

32. A method, comprising:
providing a composition to a subject by an oral, nasopharyngeal, oropharyngeal, pulmonary, buccal, sublingual, mucosal, vaginal, or rectal route, said composition comprising a first targeting element that specifically binds to a first cell surface component, and a second targeting element that specifically binds to a second component of the cell with a medically-relevant moiety,
whereby said first and second targeting elements promote bioavailability of all or a portion of the composition to into or across a cells within said subject, in comparison to compositions lacking one or both of said first and second targeting elements.

33. A method according to claim 32, wherein said medically-relevant moiety is a therapeutic moiety.

34. A method according to claim 32, wherein said first targeting element binds to pIgR.

35. A method according to claim 32, wherein said first targeting element binds to a non-secretory component region of pIgR.

36. A method according to claim 32, wherein said first targeting element binds to B region of pIgR

37. A method according to claim 32, wherein said first targeting element binds to pIgR stalk.

38. A method according to claim 32, wherein said first targeting element is selected from the group consisting of a polypeptide, a recombinant polypeptide, an antibody, an antibody fragment, a single-chain variable region fragment, a small molecule, an oligonucleotide, an oligosaccharide, a polysaccharide, a cyclic polypeptide, a peptidomimetic, and an aptamer.

39. A method according to claim 32, wherein said cell surface component is selected from the group consisting of pIgR, pIgR stalk, transferrin receptor, apo-transferrin, holo-transferrin, vitamin B12 receptor, FcRn, an integrin, Flt-1, Flk-1, Flt-4, a GPI-linked protein, a scavenger receptor, and low density lipoprotein receptor.

40. A method according to claim 32, wherein said second targeting element is selected from the group consisting of a polypeptide, a recombinant polypeptide, an antibody, an antibody fragment, a single-chain variable region fragment, a small molecule, an oligonucleotide, an oligosaccharide, a polysaccharide, a cyclic polypeptide, a peptidomimetic, and an aptamer.

41. A method according to claim 32, wherein said second component is selected from the group consisting of pIgR, pIgR stalk, transferrin receptor, apo-transferrin, holo-transferrin, vitamin B12 receptor, FcRn, an integrin, Flt-1, Flk-1, Flt-4, a GPI-linked protein, a scavenger receptor, and low density lipoprotein receptor.

42. A method according to claim 32, wherein said therapeutic moiety is selected from the group consisting of a polypeptide, a recombinant polypeptide, an antibody, an antibody fragment, a single-chain variable region fragment, a small molecule, an oligonucleotide, an oligosaccharide, a polysaccharide, a cyclic polypeptide, a peptidomimetic, and an aptamer.

43. A method according to claim 33, wherein said medically-relevant moiety is an antibody, and said antibody is covalently or noncovalently bound to one or more first targeting moieties that are single-chain variable region fragments that specifically bind to pIgR.

44. A method according to claim 32, wherein the first targeting element is an antibody or fragment thereof or a single-chain variable region fragment, and said first targeting element is covalently or noncovalently bound to a second targeting element selected from the group consisting of transferrin, antibody to transferrin, antibody to transferrin receptor, and transferrin binding peptide.

45. A method according to claim 32, wherein said composition comprises a particulate structure selected from the group consisting of a nanoparticle, a microparticle, a nanocapsule, and a microcapsule.

46. A method according to claim 32, wherein said composition is provided by a pulmonary route.

47. A method according to claim 32, wherein said composition is provided by an oral route.
